(19) United States
(12) Patent Application Publication
(10) Pub. No.: US 2026/0364147 A1
(43) Pub. Date: Nov. 19, 2026

(54) LOW DOSE HUMAN INTERLEUKIN-2 FOR THE TREATMENT OF AMYOTROPHIC LATERAL SCLEROSIS IN A SUBGROUP OF PATIENTS

(71) Applicants: Centre Hospitalier Universitaire De Nimes, Nimes Cédex 9 (FR); The University of Sussex, Brighton East Sussex (GB); Humanitas Mirasole Spa, Rozzano (IT); King's College London, Londres (GB); The University of Sheffield, Sheffield (GB); Queen Mary University of London, London (GB)

(72) Inventors: Gilbert BENSIMON, Villejuif (FR); Peter Nigel LEIGH, Robertsbridge (GB); Timothy TREE, London East Dulwich (GB); Ammar AL-CHALABI, Market Harborough, Leicestershire (GB); Cecilia GARLANDA, Milano (IT); Massimo LOCATI, Rozzano (IT); Janine KIRBY, Doncaster (GB); Pamela SHAW, Hope Valley, Derbyshire (GB); Andrea MALASPINA, London (GB)

(73) Assignees: Centre Hospitalier Universitaire De Nimes, Nimes Cédex 9 (FR); The University of Sussex, Brighton East Sussex (GB); Humanitas Mirasole Spa, Rozzano (IT); King's College London, Londres (GB); The University of Sheffield, Sheffield (GB); Queen Mary University of London, London (GB)

(21) Appl. No.: 19/135,563

(22) PCT Filed: Dec. 5, 2023
(86) PCT No.: PCT/EP2023/084388

§ 371 Date: Jun. 4, 2025

(30) Foreign Application Priority Data
Dec. 5, 2022 (EP) 22306791.9

(51) Int. Cl. A61K 38/20; A61K 31/428; A61P 25/28; G01N 33/68

(52) U.S. Cl. CPC A61K 38/2013; A61K 31/428; A61P 25/28; G01N 33/6896; G01N 2470/04; G01N 2800/28; G01N 2800/52

(57) ABSTRACT

The present invention is in the field of amyotrophic lateral sclerosis (ALS) and relates to human interleukin-2 (IL-2) for use in the treatment of amyotrophic lateral sclerosis in a human subject, wherein each dose of human IL-2 administered to said subject CA is between 0.1×10⁶ to 3×10⁶ international units (IUJ) and the subject has a low to medium concentration of p-NFH in cerebrospinal fluid (CSF p-NFH) or a low to medium concentration of NFL or NFM in cerebrospinal fluid, blood, serum or plasma before human IL-2 administration. The invention also relates to medical uses where the CSF p-NFH or CSF, blood, serum or plasma NFL or NFM concentration is used to select the best administration scheme or as a biomarker for stratified randomization of a cohort of ALS patients in the context of a clinical trial assessing the therapeutic efficiency of a candidate ALS treatment.

### TECHNICAL FIELD OF THE INVENTION

The present invention is in the field of amyotrophic lateral sclerosis (ALS). It relates to human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the subject has a low to medium concentration of p-NFH in cerebrospinal fluid (CSF p-NFH) or a low to medium concentration of NFL or NFM in cerebrospinal fluid, blood, serum or plasma before human IL-2 administration. The invention also relates to medical uses where the CSF p-NFH or CSF, blood, serum or plasma NFL or NFM concentration is used to select the best administration scheme or as a biomarker for stratified randomization of a cohort of ALS patients in the context of a clinical trial assessing the therapeutic efficiency of a candidate ALS treatment.

### BACKGROUND ART

Amyotrophic Lateral Sclerosis (ALS) is a fatal neuromuscular disease, clinically characterized by relentlessly progressive weakness, muscle wasting and loss of motor function. Despite the introduction of riluzole two decades ago (Bensimon G et al N Engi J Med 1994; 330: 585-91), subsequent trials have failed to deliver more effective disease-modifying remedies.

The many drug development failures in ALS are mainly related to the fact that ALS is a complex disease, for which there is no validated predictive animal model. In particular, while transgenic mutant SOD1 (mSOD1) mice are often used as an ALS animal model, all positive results obtained in this model have failed to translate into efficient remedies for human subjects with ALS. This may reflect the substantial difference between a SOD1-driven pathology in this animal model from the recognized TDP-43 pathology in the prevalent human sporadic ALS. (DiBernardo A B et al. Biochimica et Biophysica Acta 1762 (2006) 1139-1149; van den Berg L H et al. Neurology 2019; 92:e1610-e1623). ALS is also characterized by its heterogeneity, with high variability of etiopathogenic, genetic, pathological, clinical, cognitive and dynamic factors (Beghi E et al. Amyotroph Lateral Scler. 2011 January; 12(1):1-10).

Neuro-inflammatory processes are prominent pathological features in subjects living with ALS. Microglial cell activation is evidenced in the pathology of ALS at all disease stages (Troost D et al. Neuropathol Appl Neurobiol 1990; 16: 401-10; Kawamata T et al. Am J Pathol 1992; 140: 691; Engelhardt J I et al. Arch Neurol 1993; 50: 30-6; Thonhoff J R et al. Curr Opin Neurol 2018; 31: 635-9) and in the transgenic SOD1 ALS mouse (Engelhardt J I et al. Arch Neurol 1993; 50: 30-6; McGeer P L et al. Muscle Nerve 2002; 26: 459-70; Hall E D, Oostveen J A, Gurney M E. Glia 1998; 23: 249-56), in which expression of macrophage-typical cytokines precedes clinical symptoms (Alexianu M E et al. Neurology 2001; 57: 1282-9; Hensley K et al. J Neurochem 2002; 82: 365-74). Furthermore, biomarkers of neuro-inflammation are elevated in subjects with ALS and have been shown to correlate with disease severity and predict disease progression (Gille B et al. J Neurol Neurosurg Psychiatry. 2019 December; 90(12):1338-1346; Tateishi T et al. J Neuroimmunol. 2010 May; 222(1-2):76-81).

Although evidence of a neuro-inflammatory contribution to ALS pathogenesis is compelling (Evans M C et al. Mol Cell Neurosci 2013; 53: 34-41; Zhao W et al. J Neuroimmune Pharmacol Off J Soc Neuroimmune Pharmacol 2013; 8: 888-99), most therapeutic attempts to modify the neuro-inflammatory response in the ALS clinical context have failed (Appel S H et al. Arch Neurol 1988; 45: 381; Drachman D B et al. Ann Neurol 1994; 35: 142-50; Tan E et al. Arch Neurol 1994; 51: 194; Beghi E et al. Neurology 2000; 54: 469-469; Cudkowicz M E et al. Ann Neurol 2006; 60: 22-31; Gordon P H et al. Lancet Neurol 2007; 6: 1045-53; Stommel E W et al. Amyotroph Lateral Scler 2009; 10: 393-404). Most of these trials have however targeted non-specific suppression of neuro-inflammation. Such approaches have foremost a high risk to harm subjects with ALS, where toxicity may easily outweigh a beneficial drug effect.

In this context, new approaches reinforcing physiological tolerogenic dominance within the neuroimmuno-inflammatory system without suppressing all immunity are needed. CD4+FOXP3+ regulatory T cells (Tregs) physiologically regulate immune responses, contributing to the induction and maintenance of tolerance thus preventing the onset of autoimmune and inflammatory diseases (Sakaguchi S et al. Cell 2008; 133: 775-87).

Previous studies have shown that in ALS subjects decreased levels of Tregs were correlated with increased disease severity and were predictive of disease progression and survival (Mantovani S et al. J Neuroimmunol 2009; 210: 73-9; Rentzos M et al. Acta Neurol Scand 2012; 125: 260-4; Henkel J S et al. EMBO Mol Med 2013; 5: 64-79). In addition to their decreased levels, Tregs of ALS subjects expressed lower levels of FOXP3 (Henkel J S et al. EMBO Mol Med 2013; 5: 64-79) and were shown to be dysfunctional, their dysfunction correlating with increased disease severity (Beers D R et al. JCI Insight. 2017; 2(5):e89530; Thonhoff J R et al. Curr Opin Neurol 2018; 31: 635-9). Therefore, in ALS subjects, Tregs are not only reduced in numbers but also show significant dysfunction correlated to impaired FOXP3 expression level, and their dysfunction correlates to disease severity and progression, suggesting that Treg suppressive function might be a meaningful indicator of clinical status (Thonhoff J R et al. Curr Opin Neurol 2018; 31: 635-9).

Tregs are exclusively reliant on the cytokine Interleukin 2 (IL-2) for their generation, activation and survival (Malek T R, Bayer A L. Nat Rev Immunol 2004; 4: 665-74). Moreover, contrary to human effector T-cells (Teffs), human Tregs constitutively express high levels of CD25, forming a high-affinity receptor for IL-2 and thus respond to low concentrations of IL-2, insufficient to stimulate Teffs (Dupont G. et al. Cytokine. 2014 September; 69(1):146-9). On this basis, low dose (ld) IL-2 administration has been tested and shown to induce the selective expansion of Tregs in mice and humans in Healthy Volunteers or type 1 diabetes contexts (Hartemann A et al. Lancet Diabetes Endocrinol 2013; 1: 295-305 Ito S et al. Mol Ther J Am Soc Gene Ther 2014. 22: 1388-95).

On this basis, use of ld-IL-2 has been proposed in the treatment of various auto-immune and inflammatory conditions (WO2012123381A1, WO2014023752A1, WO2016025385A1, WO2016164937A2) and several clinical trials exploring the therapeutic potential of ld-IL-2 in graft-versus-host disease (Koreth J et al. N Engi J Med 2011; 365: 2055-66), HCV-induced vasculitis (Saadoun D et al. N Engi J Med 2011; 365: 2067-77), type 1 diabetes (Hartemann A et al. Lancet Diabetes Endocrinol 2013; 1: 295-305), alopecia areata (Castela E et al. JAMA Dermatol 2014; 150: 748-51) have been reported.

In the context of ALS, it was initially claimed that Tregs from ALS human subjects have impaired endogenous responsiveness to IL-2, and that ldIL-2 was not found to alter the clinical outcome or to increase endogenous Treg numbers, in a non-blind, non-placebo-controlled trial on a very limited number of subjects (Thonhoff J R et al. Neurol Neuroimmunol Neuroinflammation 2018; 5: e465).

However, it was then found in a three-arm, randomized (1:1:1), double-blind, single-center study (IMODALS) of 2 doses of ld-IL-2 in parallel versus placebo clinical trial including 36 subjects (12 in each of the 3 treatment arms), that ldIL-2 injection to ALS subjects is (i) safe and well tolerated, and is able both to (ii) upregulate Tregs numbers and suppressive function over Teffs, (iii) downregulate inflammatory markers of disease progression (CCL2), (iv) shift monocyte polarization from M1 pro-inflammatory phenotype to M2 anti-inflammatory and tissue repair phenotype, and (v) decrease overall ALS-related cytopathic activity as evidenced by plasma NFL response to treatment, indicative of decreased axonal lesioning (Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044) and is thus an efficient treatment of ALS.

It is however well known that any complex disease is subject to interindividual variability and that no treatment is efficient in all subjects suffering from a particular disease.

There is thus a need for suitable biomarkers able to predict which ALS patients will benefit from low dose IL-2 treatment.

CSF and plasma concentrations of neurofilaments have consistently been reported as predictors of survival and disease progression (PRADO LAURA DE GODOY ROUSSEFF ET AL: “Longitudinal assessment of clinical and inflammatory markers in patients with amyotrophic lateral sclerosis”, JOURNAL OF NEUROLOGICAL SCIENCES, ELSEVIER SCIENTIFIC PUBLISHING CO, AMSTERDAM, NL, vol. 394, 4 Sep. 2018 (2018-09-04), pages 69-74). In addition, some authors have speculated that they might be useful for monitoring response to treatment, based on the fact that a neuroprotective treatment should result in a decrease of neurofilament concentrations, so that a comparison between neurofilaments concentration before and after treatment might permit to assess treatment efficiency (KOEN POESEN ET AL: “Diagnostic and Prognostic Performance of Neurofilaments in ALS”, FRONTIERS IN NEUROLOGY, vol. 9, 18 Jan. 2019 (2019-01-18)).

However, a biomarker that would permit to predict response to low dose human IL-2 treatment based on a single sample obtained before treatment is started is still lacking.

### SUMMARY OF THE INVENTION

In the context of the present invention, the inventors surprisingly found that low dose human interleukin-2 (IL-2) mainly shows therapeutic efficiency in ALS subjects having a low to medium (in particular medium) concentration of p-NFH in cerebrospinal fluid (CSF p-NFH) before human IL-2 administration (see Example 1 below).

The present invention thus relates to human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the subject has or has been selected based on a measure performed in vitro in a sample obtained before human IL-2 administration for:
a) a low to medium concentration of p-NFH in cerebrospinal fluid (CSF p-NFH) before human IL-2 administration, which is preferably lower than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA);
b) a low to medium concentration of p-NFH in cerebrospinal fluid (CSF p-NFH) before human IL-2 administration when measured by another assay (i.e. an assay other than sandwich ELISA), which is preferably a concentration of p-NFH in cerebrospinal fluid (CSF p-NFH) before human IL-2 administration lower than a CSF_p-NFH_medium-high_other-assay threshold value when measured by another assay, wherein the CSF_p-NFH_medium-high_other-assay threshold value is correlated, preferably linearly correlated, to the CSF_p-NFH_medium-high_sandwich-ELISA threshold value;
c) a low to medium concentration of NFL in cerebrospinal fluid, blood, serum or plasma (CSF, blood, serum or plasma NFL) before human IL-2 administration when measured by a specific assay, which is preferably a concentration of NFL in cerebrospinal fluid, blood, serum or plasma (CSF, blood, serum or plasma NFL) before human IL-2 administration lower than a CSF, blood, serum or plasma_NFL_medium-high_specific-assay threshold value that is correlated, preferably linearly correlated, to the CSF_p-NFH_medium-high_sandwich-ELISA threshold value; or
d) a low to medium concentration of NFM in cerebrospinal fluid cerebrospinal fluid, blood, serum or plasma (CSF, blood, serum or plasma NFM) before human IL-2 administration when measured by a specific assay, which is preferably a concentration of NFM in cerebrospinal fluid cerebrospinal fluid, blood, serum or plasma (CSF, blood, serum or plasma NFM) before human IL-2 administration lower than a CSF, blood, serum or plasma_NFM_medium-high_specific-assay threshold value that is correlated, preferably linearly correlated, to the CSF_p-NFH_medium-high_sandwich-ELISA threshold value.

The present invention also relates to human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the administration scheme is adapted based on the subject's:
a) CSF pNFH concentration,
b) CSF, blood, serum or plasma NFL concentration, or
c) CSF, blood, serum or plasma NFM concentration.

The present invention also relates to the use of the CSF pNFH concentration; the CSF, blood, serum or plasma NFL concentration; or the CSF, blood, serum or plasma NFM concentration of a cohort of ALS patients included in a clinical trial intended to assess the therapeutic efficiency of a candidate ALS treatment (including low dose human IL-2) as a biomarker for stratification of the cohort of ALS patients into three subgroups with low, medium or high concentration, and separate randomization of each subgroup into the candidate ALS treatment or placebo arm.

### DESCRIPTION OF THE FIGURES

FIG. 1. Scheme of MIROCALS clinical trial set-up.

FIG. 2. CSF-pNFH at randomisation—distribution and proposed cut-off. Normality test Parametric Shapiro-Wilk test: W=0.69, p<2.2 E-16—Non normal. Mode test Ameijeiras-Alonso et al. (2019): Critical bandwidth 0.029688, p=0.808—number of modes is >1.

FIG. 3. Survival probability in time of the three groups of patients with the best cut-off of 3700 pg/ml for distinguishing medium and high CSF pNFH concentration.

FIG. 4. Kaplan Meier survival curves of placebo and ld IL2 (low dose IL-2) in the Medium (750-3700 pg/ml) CSF-pNFH representing 70% of overall ITT population.

FIG. 5. Kaplan Meier survival curves of placebo and ld IL2 (low dose IL-2) in the Low (LLOQ)+Medium populations (CSF-pNFH<3700 pg/ml) representing 79% of the overall ITT population.

FIG. 6. Kaplan Meier survival curves of placebo and ld IL2 (low dose IL-2) in the High(>3700 pg/ml) CSF-pNFH population representing 21% of overall ITT population.

FIG. 7. Correlation of CSF pNFH concentration with CSF NFL concentration.

### DETAILED DESCRIPTION OF THE INVENTION

In the context of the present invention, the inventors surprisingly found that low dose human interleukin-2 (IL-2) mainly shows therapeutic efficiency in ALS subjects having a low to medium (in particular medium) concentration of p-NFH in cerebrospinal fluid (CSF p-NFH) before human IL-2 administration (see Example 1 below).

### Medical Use and Methods of Treatment in a Subgroup of Als Patients

The present invention thus relates to human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the subject has or has been selected based on a measure performed in vitro in a sample obtained before human IL-2 administration for:
a) a low to medium concentration of p-NFH in cerebrospinal fluid (CSF p-NFH) before human IL-2 administration, preferably lower than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA);
b) a low to medium concentration of p-NFH in cerebrospinal fluid (CSF p-NFH) before human IL-2 administration when measured by another assay (i.e. an assay other than sandwich ELISA), preferably a concentration of p-NFH in cerebrospinal fluid (CSF p-NFH) before human IL-2 administration lower than a CSF_p-NFH_medium-high_other-assay threshold value when measured by another assay, wherein the CSF_p-NFH_medium-high_other-assay threshold value is linearly correlated to the CSF_p-NFH_medium-high_sandwich-ELISA threshold value;
c) a low to medium concentration of NFL in cerebrospinal fluid, blood, serum or plasma (CSF, blood, serum or plasma NFL) before human IL-2 administration when measured by a specific assay, preferably a concentration of NFL in cerebrospinal fluid, blood, serum or plasma (CSF, blood, serum or plasma NFL) before human IL-2 administration lower than a CSF, blood, serum or plasma_NFL_medium-high_specific-assay threshold value that is linearly correlated to the CSF_p-NFH_medium-high_sandwich-ELISA threshold value; or
d) a low to medium concentration of NFM in cerebrospinal fluid cerebrospinal fluid, blood, serum or plasma (CSF, blood, serum or plasma NFM) before human IL-2 administration when measured by a specific assay, preferably a concentration of NFM in cerebrospinal fluid cerebrospinal fluid, blood, serum or plasma (CSF, blood, serum or plasma NFM) before human IL-2 administration lower than a CSF, blood, serum or plasma_NFM_medium-high_specific-assay threshold value that is linearly correlated to the CSF_p-NFH_medium-high_sandwich-ELISA threshold value.

In embodiment a) above, a sandwich ELISA assay is used for measuring CSF p-NFH concentration, based on the level of which the ALS subject is or not selected for low dose human IL-2 administration. In embodiment b), CSF p-NFH concentration is measured by another assay than sandwich ELISA but the measures of the two assays are linearly correlated and the threshold value useful for selecting or not the ALS subject for low dose human IL-2 administration is correlated to the threshold value useful in embodiment a) when the CSF p-NFH concentration is measured using a sandwich ELISA assay. In embodiments c) and d), NFL or NFM concentration in CSF, blood, serum or plasma is measured, either using a sandwich ELISA assay or using another specific assay (different from sandwich ELISA). In any case, as p-NFH, NFL and NFM concentrations are correlated and values obtained using various assays are also correlated, the threshold value useful for selecting or not the ALS subject for low dose human IL-2 administration is still correlated to the threshold value useful in embodiment a) when the CSF p-NFH concentration is measured using a sandwich ELISA assay.

The present invention also relates to the use of human interleukin-2 (IL-2) for the manufacture of a drug for use in the treatment of amyotrophic lateral sclerosis in a human subject, wherein each dose of human IL-2 administered to said subject during said treatment is between 0.1×10⁶ to 3×10⁶ international units (IU) and the subject is as disclosed in the 1^{st} preceding paragraph.

The present invention also relates the use of human interleukin-2 (IL-2) in the treatment of amyotrophic lateral sclerosis in a human subject, wherein each dose of human IL-2 administered to said subject is between 0.1×10⁶ to 3×10⁶ international units (IU) and the subject is as disclosed in the 2^{nd} preceding paragraph.

The present invention also relates to a pharmaceutical composition comprising human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the subject is as disclosed in the 3^{rd} preceding paragraph.

The present invention also relates to a method for treating amyotrophic lateral sclerosis in a human subject in need thereof, comprising administering to said human subject human interleukin-2 (IL-2), wherein each dose of human IL-2 administered to said subject is between 0.1×10⁶ to 3×10⁶ international units (IU) and the subject is as disclosed in the 4^{th} preceding paragraph.

The present invention also relates to a method for treating amyotrophic lateral sclerosis in a human subject in need thereof, comprising:
a) Measuring the concentration of p-NFH in a cerebrospinal fluid sample of the subject, or the concentration of NFL in a CSF, blood, serum or plasma sample of the subject, or the concentration of NFM in a CSF, blood, serum or plasma sample of the subject; and
b) Administering to the subject:
   i) human interleukin-2 (IL-2), wherein each dose of human IL-2 administered to said subject is between 0.1×10⁶ to 3×10⁶ international units (IU) and optionally riluzole if:
      the concentration of p-NFH in cerebrospinal fluid (CSF p-NFH) measured in step a) is low to medium, preferably lower than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA) or lower than a CSF_pNFH_medium-high_other-assay threshold value that is linearly correlated to the CSF_p-NFH_medium-high_sandwich-ELISA threshold value or lower than a CSF, blood, serum or plasma_NFL_medium-high_specific-assay threshold value that is linearly correlated to the CSF_p-NFH_medium-high_sandwich-ELISA threshold value,
      the concentration of NFL in cerebrospinal fluid, blood, serum or plasma measured in step a) when measured by a specific assay is low to medium, preferably the concentration of NFL in cerebrospinal fluid, blood, serum or plasma (CSF, blood, serum or plasma NFL) before human IL-2 administration is lower than a CSF, blood, serum or plasma_NFL_medium-high_specific-assay threshold value that is linearly correlated to the CSF_p-NFH_medium-high_sandwich-ELISA threshold value, or
      the concentration of NFM in cerebrospinal fluid, blood, serum or plasma measured in step a) when measured by a specific assay is low to medium, preferably the concentration of NFM in cerebrospinal fluid, blood, serum or plasma (CSF, blood, serum or plasma NFM) before human IL-2 administration is lower than a CSF, blood, serum or plasma_NFL_medium-high_specific-assay threshold value that is linearly correlated to the CSF_p-NFH_medium-high_sandwich-ELISA threshold value; or
   ii) riluzole and optionally another treatment different from human IL-2 if:
      the concentration of p-NFH in cerebrospinal fluid (CSF p-NFH) measured in step a) is high, preferably higher than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA) or higher than a CSF_p-NFH_medium-high_other-assay threshold value that is linearly correlated to the CSF_p-NFH_medium-high_sandwich-ELISA threshold value or higher than a CSF, blood, serum or plasma_NFL_medium-high_specific-assay threshold value that is linearly correlated to the CSF_p-NFH_medium-high_sandwich-ELISA threshold value,
      the concentration of NFL in cerebrospinal fluid, blood, serum or plasma measured in step a) when measured by a specific assay is high, preferably the concentration of NFL in cerebrospinal fluid, blood, serum or plasma (CSF, blood, serum or plasma NFL) before human IL-2 administration is higher than a CSF, blood, serum or plasma_NFL_medium-high_specific-assay threshold value that is linearly correlated to the CSF_p-NFH_medium-high_sandwich-ELISA threshold value, or
      the concentration of NFM in cerebrospinal fluid, blood, serum or plasma measured in step a) when measured by a specific assay is high, preferably the concentration of NFM in cerebrospinal fluid, blood, serum or plasma (CSF, blood, serum or plasma NFM) before human IL-2 administration is lower higher a CSF, blood, serum or plasma_NFL_medium-high_specific-assay threshold value that is linearly correlated to the CSF_p-NFH_medium-high_sandwich-ELISA threshold value.

### Treated Human Subject in Medical Uses and Treatment Methods

In the MIROCALS clinical trial, the inventors have found that low dose IL-2 treatment is particularly efficient for treating ALS in subjects who have a low or medium (lower than 3700 pg/ml with the specifically used sandwich ELISA assay) concentration of p-NFH in cerebrospinal fluid (CSF p-NFH) before human IL-2 administration when measured by a specific sandwich enzyme-linked immunosorbent assay (ELISA).

Therefore, in an embodiment of the invention, the treated human subject has or has been selected based on a measure performed in vitro in a sample obtained before human IL-2 administration for a low or medium concentration of p-NFH in cerebrospinal fluid (CSF p-NFH) before human IL-2 administration when measured by a sandwich ELISA assay. Preferably, the treated human subject has been selected based on a measure performed in vitro in a sample obtained before human IL-2 administration for a low or medium concentration of p-NFH in cerebrospinal fluid (CSF p-NFH) before human IL-2 administration when measured by a sandwich ELISA assay (i.e. CSF p-NFH has been measured in a CSF sample obtained from the human subject before human IL-2 administration by a sandwich ELISA assay and found to be low or medium, i.e. lower than a CSF_p-NFH_medium-high_sandwich-ELISA threshold value).

“p-NFH” or “pNFH” or “pNF-H” or “pNfH” or “phosphorylated NFH” or “phosphorylated neurofilament heavy subunit” or “phosphorylated neurofilament heavy chain” or “phosphorylated neurofilament H” refers to a cytoskeletal structural protein released as a result of axonal damage into the cerebrospinal fluid (CSF), and subsequently into the blood. Neurofilaments are neuron-specific cytoskeletal proteins with a characteristic diameter of 8-10 nm, and are members of the intermediate filament family. Neurofilaments contain three distinct subunits named according to the molecular mass of their subunits as light (NFL), medium (NFM) and heavy chain (NFH). Post-translationaL modifications like phosphorylation and O-glycosylation are crucial for neurofilaments aggregation, especially in NFM and NFH. In particular, NFH is about 200 kDa and contains unusual multiple repeated sequence lysine-serine-proline (KSP), and in axonal neurofilaments essentially all serine residues are heavily phosphorylated. Phosphorylation is represented by small case letter “p” before the specific neurofilament. Therefore, “p-NFH” or “pNFH” represents the phosphorylated form of neurofilament heavy chain. Because phosphorylated forms of NFH (pNFH) are quite resistant to proteases, pNFH released from damaged and diseased axons should remain in fluid undegraded.

pNFH may be found in “cerebrospinal fluid” (also referred to as “CSF”), a clear, colorless body fluid found within the tissue that surrounds the brain and spinal cord of all vertebrates. A sample of CSF can be taken from around the spinal cord via lumbar puncture.

“NFL” or “NF-L” or “NfL” or “neurofilament light subunit” or “neurofilament light chain” or “neurofilament L” refers to the light chain of neurofilaments released as a result of axonal damage into the cerebrospinal fluid (CSF), and subsequently into the blood. NFL is about 68 kDa.

“NFM” or “NF-M” or “NfM” or “neurofilament medium subunit” or “neurofilament medium chain” or “neurofilament M” refers to the medium chain of neurofilaments released as a result of axonal damage into the cerebrospinal fluid (CSF), and subsequently into the blood. NFM is about 150 kDa.

Assays for Measuring the Concentration of p-NFH in CSF

### Sandwich ELISA Assay

The concentration of p-NFH in CSF is measured using a sandwich enzyme-linked immunosorbent assay (ELISA).

ELISA (which stands for enzyme-linked immunosorbent assay) is a technique to detect the presence of antigens and quantify them in biological samples. An ELISA, like other types of immunoassays, relies on antibodies to detect a target antigen using highly specific antibody-antigen interactions.

Such an assay comprises several successive steps:
Capture step 1) comprising:
   1a) adding a CSF sample to one or more wells of a plate pre-coated with a first anti-human p-NFH antibody recognizing a first epitope on human p-NFH,
   1b) incubating the plate for an appropriate duration at a suitable temperature so that human p-NFH present in the CSF sample is captured by the first anti-human p-NFH antibody coated on the plate,
   1c) removing the CSF sample from the wells and washing the wells with a washing solution in order to remove compounds that are not captured by the first anti-human p-NFH antibody;
Detection step 2) comprising:
   2a) detecting captured human pNFH by adding a detection solution comprising a second anti-human p-NFH antibody recognizing a second epitope on human p-NFH, wherein said second anti-human p-NFH antibody is labelled or labelable with a directly or indirectly detectable molecule to each well,
   2b) incubating the plate for an appropriate duration at a suitable temperature so that the second human p-NFH antibody binds to captured human pNFH,
   2c) removing the detection solution from the wells and washing the wells with a washing solution in order to remove from the plate the second anti-human p-NFH antibody that is not bound to captured human pNFH,
   2d) when the second human p-NFH antibody is labelable with a directly or indirectly detectable molecule, adding a revealing solution comprising an affinity molecule that comprises a directly or indirectly detectable molecule and specifically binds to the second human p-NFH antibody, incubating the plate for an appropriate duration at a suitable temperature so that the second human p-NFH antibody binds to the affinity molecule, removing the revealing solution and washing the wells with a washing solution in order to remove the unbound affinity molecule from the plate;
Measure step 3) comprising: directly or indirectly measuring the amount of labelled antibody or of labelable antibody complexed to the affinity molecule present in each well of the plate; and
Calculation step 4) comprising: calculating the concentration of pNFH in the CSF sample based on the results of measure step 3).

### Capture Step 1)

In capture step 1), a CSF sample is added to one or more wells of a plate pre-coated with a first anti-human p-NFH antibody recognizing a first epitope on human p-NFH (also referred to as the capture antibody), the plate is incubated for an appropriate duration at a suitable temperature so that human p-NFH present in the CSF sample is captured by the first anti-human p-NFH antibody coated on the plate, then the CSF sample is removed from the wells and the wells are washed with a washing solution in order to remove compounds that are not captured by the first anti-human p-NFH antibody.

The CSF sample added in sub-step 1a) may have been previously diluted (for instance by a factor comprised between 2 and 5, such as a factor 2, 3, 4 or 5) before addition to the well(s) of the plate.

Preferably, the same (optionally diluted) CSF sample is added to at least 2 (such as two or three) wells of the plate (the measure is then done in duplicate or triplicate).

The plate is generally a multiwell plate, such as a 96-well or 384-well plate. It may be made of any suitable material, such as polystyrene.

The first anti-human p-NFH antibody may be any antibody specifically recognizing human p-NFH. It may notably be polyclonal or monoclonal. The first anti-human p-NFH antibody is preferably not labelled by a directly or indirectly detectable molecule, as defined below in detection step 2).

In addition to being pre-coated with the first anti-human p-NFH antibody, the plate has preferably been previously blocked using a blocking solution, which prevents antibodies or other proteins from adsorbing to the plate during subsequent steps. A blocking solution is a solution of irrelevant protein, mixture of proteins, or other compound that passively adsorbs to all remaining binding surfaces of the plate. The blocking solution is effective if it improves the sensitivity of an assay by reducing background signal and improving the signal-to-noise ratio. If a complete sandwich ELISA kit is not used, then several different blocking solutions should be tested for the highest signal to noise ratio in the assay. Many factors can influence nonspecific binding, including various protein-protein interactions unique to the samples and antibodies involved. The most important parameter when selecting a blocking solution is the signal to noise ratio, which is measured as the signal obtained with a sample containing the target analyte as compared to that obtained with a sample without the target analyte. An optimal concentration of blocking solution should also be determined. However, commercial kits comprising an already optimized blocking solution and information regarding its optimal concentration or pre-coated and pre-blocked plates are commercially available, such as the Biovendor Human Phosphorylated Neurofilament H ELISA kit (BioVendor, cat #RD191138300R) used in MIROCALS clinical trial.

The plate is then incubated in sub-step 1b) for an appropriate duration (e.g. 30 to 120 minutes, 45 to 90 minutes, such as about 60 minutes) at a suitable temperature (generally room temperature, i.e. between 18 °C and 25 °C) so that human p-NFH present in the CSF sample is captured by the first anti-human p-NFH antibody coated on the plate.

Finally, in sub-step 1c), the CSF sample is removed from the wells and the wells are washed with a washing solution in order to remove compounds that are not captured by the first anti-human p-NFH antibody. A washing step is useful to remove non-bound reagents and decrease background, thereby increasing the signal to noise ratio. Insufficient washing may cause high background, while excessive washing might result in decreased sensitivity caused by elution of the antibody and/or antigen from the well. Washing is generally performed either in a physiologic buffer such as Tris-buffered saline (TBS) or phosphate-buffered saline (PBS) or in a diluted bocking solution, to which a detergent such as 0.05% Tween-20 is added to the buffer to help remove nonspecifically bound material. Including the blocking solution and adding a detergent in washing solution helps to minimize background in the assay. For best results, high-purity detergents should be used to prevent introduction of impurities that will interfere with the assay such enzyme inhibitors or peroxides. In step 2), the wells may be washed several times with the washing solution, such as 2 to 5 times, preferably 2 or 3 times.

### Detection Step 2)

In detection step 2), captured human pNFH is detected by firstly adding a detection solution comprising a second anti-human p-NFH antibody recognizing a second epitope on human p-NFH (also referred to as the detection antibody), wherein said second anti-human p-NFH antibody is labelled or labelable with a directly or indirectly detectable molecule (sub-step 2a)).

The second anti-human p-NFH antibody may be any antibody specifically recognizing a second epitope on human p-NFH, which is preferably different from and non-overlapping with the first epitope recognized by the first anti-human p-NFH antibody used for capture. It may notably be polyclonal or monoclonal. Pairs of anti-human p-NFH antibodies recognizing non-overlapping epitopes on human p-NFH are commercially available. For instance, the Biovendor Human Phosphorylated Neurofilament H ELISA kit (BioVendor, cat #RD191138300R) used in MIROCALS clinical trial comprises a chicken polyclonal anti-human p-NFH antibody as first capture antibody, and a rabbit polyclonal anti-human p-NFH antibody as a second detection antibody.

The second anti-human p-NFH antibody may be labelled or labelable with a directly or indirectly detectable molecule.

An antibody is said to be “labelled” or “directly labelled” with a directly or indirectly detectable molecule when the antibody is covalently bound to the directly or indirectly detectable molecule. Conversely, an antibody is said to be “labelable” with a directly or indirectly detectable molecule when the antibody may be complexed with (i.e. non covalently bound to) an affinity molecule that specifically binds to the indirectly labelled antibody and comprises the directly or indirectly detectable molecule. For instance, an antibody covalently bound to a fluorophore is a labelled antibody, whereas a biotinylated antibody that maybe complexed with (i.e. non covalently bound to) fluorescent avidin or streptavidin is a labelable antibody. Other examples of labelled antibodies include antibodies covalently bound to a colored molecule, a luminescent molecule, or an enzyme able to convert a substrate into a fluorescent, colored or luminescent molecule (such as HRP or AP). Another example of labelable antibody is an antibody that may be specifically bound by a labelled third antibody (for instance an antibody specifically binding to constant antibody regions of the species of the second detection antibody, in which case the second anti-human pNFH antibody added in step 2) has to be from a species different from the species of the first human pNFH antibody added in step 1)). An “indirectly labelled” antibody refers to a labelable antibody complexed to the affinity molecule comprising a directly or indirectly detectable molecule.

Labelable antibodies are often used in ELISA assays and are preferred here, because:
A wide variety of labeled secondary antibodies are available commercially;
Indirect antibody labelling is versatile because many primary antibodies can be made in one species and the same labeled secondary antibody can be used for detection;
Maximum immunoreactivity of the primary antibody is retained because it is not labeled;
Sensitivity is increased because each primary antibody contains several epitopes that can be bound by the labeled secondary antibody, allowing for signal amplification; and
Different detection methods can be used with the same primary antibody (colorinetric, chemituminescent, etc.).

In the Biovendor Human Phosphorylated Neurofilament H ELISA kit (BioVendor, cat #RD191138300R) used in MIROCALS clinical trial, the second rabbit anti-human pNFH antibody is labelable with HRP through binding to a third HRP-conjugated anti-rabbit antibody.

A molecule is said to be “directly detectable” when its presence may be detected and its amount may be measured directly, without any preceding step. For instance, a fluorophore, a colored molecule, or a luminescent molecule is a directly detectable molecule.

A molecule is said to be “indirectly detectable” when its presence may be detected and its amount may be measured only indirectly, necessitating one or more additional step(s) before detection/measure. For instance, a fluorogenic molecule, a chromogenic molecule, or an enzyme able to convert a substrate into a fluorescent, colored or luminescent compound (e.g. horseradish peroxidase also referred to as “HRP”, or alkaline phosphatase also referred to as “AP”) are indirectly detectable molecules.

Indirectly detectable molecules and in particular enzymes able to convert a substrate substrate into a fluorescent, colored or luminescent compound (notably horseradish peroxidase also referred to as “HRP”, or alkaline phosphatase also referred to as “AP”) may be particularly useful as they permit distinct types of measuring techniques in step 5), depending on the specific substrate added to the wells.

The plate is then incubated for an appropriate duration (e.g. 30 to 120 minutes, 45 to 90 minutes, such as about 60 minutes) at a suitable temperature (generally room temperature, i.e. between 18 °C and 25 °C) so that the second human p-NFH antibody binds to captured human pNFH (sub-step 2b)).

The detection solution is the removed from the wells and the wells are washed with a washing solution (same as in step 1c)) in order to remove from the plate the second anti-human p-NFH antibody that is not bound to captured human pNFH.

When the second anti-human p-NFH antibody is a labelled antibody, then detection step 2) stops here.

However, when the second anti-human p-NFH antibody is a labelable antibody, then step 2) further comprises sub-step 2d), which comprises adding a revealing solution comprising an affinity molecule that comprises a directly or indirectly detectable molecule and specifically binds to the second human p-NFH antibody, incubating the plate for an appropriate duration at a suitable temperature so that the second human p-NFH antibody binds to the affinity molecule, removing the revealing solution and washing the wells with a washing solution in order to remove the unbound affinity molecule from the plate. In case of a biotinylated second anti-human p-NFH antibody, the affinity molecule may be an avidin/streptavidin molecule conjugated to a directly or indirectly detectable molecule. In another embodiment, the affinity molecule may be a third antibody specifically binding to the second anti-human pNFH antibody.

### Measure Step 3)

In measure step 3), the amount of labelled antibody or of labelable antibody complexed to the affinity molecule present in each well of the plate is directly or indirectly measured.

The type of measuring technique/apparatus used in measure step 3) will depend on the type of detectable molecule used. In the case of a colored molecule, a standard absorbance plate reader may be used. For a fluorescent molecule, a fluorometer will be used. And for a luminescent molecule, a luminometer plate reader will be used.

Depending whether a directly or indirectly detectable molecule is used, the measuring either can be done directly after the end of detection step 2) (case of a directly detectable molecule), or the measuring is made after a preliminary sub-step of transforming the indirectly detectable molecule into a directly detectable molecule (case of an indirectly detectable molecule).

For instance, when the second anti-human p-NFH antibody is a biotinylated antibody, fluorescent avidin/streptavidin may be added, the plate incubated for an appropriate duration (e.g. 5 to 30 minutes, 10 to 20 minutes, such as about 15 minutes) at a suitable temperature (generally room temperature, i.e. between 18 °C and 25 °C) and washed before detection of fluorescence using a fluorometer.

In another example, when the second anti-human p-NFH antibody is directly or indirectly Labelled by an enzyme able to convert a fluorogenic compound, a chromogenic compound or a precursor of a chemiluminescent compound into a fluorescent, colored or chemiluminescent compound (e.g. HRP or AP), a fluorogenic compound, a chromogenic compound or a precursor of a chemiluminescent compound that may be converted to a fluorescent, colored or chemiluminescent compound by the enzyme may be added, the plate incubated for an appropriate duration (e.g. 5 to 30 minutes, 10 to 20 minutes, such as about 15 minutes) at a suitable temperature (generally room temperature, i.e. between 18 °C and 25 °C) and washed before detection of fluorescence using a fluorometer, color by a standard absorbance plate reader, or light by a luminometer plate reader. Examples of fluorogenic compounds, chromogenic compounds or precursors of chemiluminescent compounds that may be converted by an enzyme such as HRP or AP into a fluorescent, colored or chemiluminescent compound are known in the art and commercially available. For instance, commonly used chromogenic HRP substrates include 3,3′,5,5′-tetramethylbenzidine (TMB), 2,2′-azino-di-[3-ethylbenzthiazoline-6-sulfonic acid](ABTS), and o-phenylenediamine dihydrochloride (OPD), and a commonly used chromogenic AP substrate is p-Nitrophenyl Phosphate (pNPP). Commonly used chemiluminescent HRP substrates include SuperSignal™ ELISA Pico Chemiluminescent Substrate and SuperSignal™ ELISA Femto Substrate, and commonly used chemiluminescent AP substrate include CSPD™, CDP-Star™ and DynaLight™ Substrate with RapidGlow™ Enhancer. Commonly fluorogenic HRP substrates include 3-(pHydroxyphenyl)propionic acid (HPPA), 10-Acetyl-3,7-dihydroxyphenoxazine (ADHP, also known as Amplex™ Red or Ampliflu™ Red), QuantaBlu™ (Emax/Amax=420/325) Fluorogenic Substrate, QuantaBlu™ (Emax/Amax=420/325) Kinetic Fluorogenic Substrate, QuantaRed™ (Emax/Amax=585/570) Enhanced Chemifluorescent HRP Substrate, and Amplex™ UltraRed substrate; and fluorogenic AP substrates include 4-Methylumbelliferyl Phosphate (MUP) and fluorescein diphosphate (FDP).

### Calculation Step 4)

In calculation step 4), the concentration of pNFH in the CSF sample is calculated based on the results of measure step 3). This is usually performed from regression function of a standard curve run on the same plate. In this case, in addition to adding a CSF sample to one or more wells of the plate in step 1), cascading dilutions of a control sample containing a known concentration of pNFH are also added to one or more wells (generally at least 2, such as 2 or 3 wells), and steps 2) and 3) are performed for all wells of the plate. The measures obtained for the wells corresponding to cascading dilutions of a control sample containing a known concentration of pNFH permit to generate a standard curve, based on which the concentration of the test CSF sample may then be calculated. When the CSF sample has been diluted before being added to the plate wells, the concentration calculated from the standard curve must then be multiplied by its dilution factor.

### Preferred Sandwich ELISA Assays

The sandwich ELISA assay used for measuring the pNFH concentration in the CSF sample of the subject to be treated may preferably comprise the following steps:
Capture step 1) comprising:
   1a1) diluting the CSF sample by a factor between 2 and 4 (preferably 3),
   1a) adding the diluted CSF sample to 2 or 3 wells of a multiwell plate (preferably a 96-well or 384-well polystyrene plate) pre-coated with a first anti-human p-NFH antibody from a first species recognizing a first epitope on human p-NFH and blocked with a blocking solution;
   1b1) adding each of several cascading dilutions of a control sample containing a known concentration of pNFH to 2 or 3 wells of the multiwell plate,
   1b) incubating the plate for 30 to 120 minutes (preferably about 60 minutes) at room temperature (between 18 °C and 25 °C) so that human p-NFH present in the diluted CSF sample or in the cascading dilutions of control sample is captured by the first anti-human p-NFH antibody coated on the multiwell plate;
   1c) removing the diluted CSF sample or the cascading dilutions of control sample from the wells and washing the wells 2 to 4 times (preferably 3 times) with a washing solution in order to remove compounds that are not captured by the first anti-human p-NFH antibody;
Detection step 2) comprising:
   2a) detecting captured human pNFH by adding to the wells a detection solution comprising a second labelable anti-human p-NFH antibody from a second different species recognizing a second epitope on human p-NFH that is different from the first epitope recognized by the first anti-human p-NFH antibody,
   2b) incubating the plate for 30 to 120 minutes (preferably about 60 minutes) at room temperature (between 18 °C and 25 °C) so that the second labelable human p-NFH antibody binds to captured human pNFH,
   2c) removing the detection solution from the wells and washing the wells 2 to 4 times (preferably 3 times) with a washing solution in order to remove from the plate the second anti-human p-NFH antibody that is not bound to captured human pNFH, and
   2d) adding a revealing solution comprising a third HRP-conjugated antibody specifically binding to the second labelable anti-human p-NFH antibody, incubating the plate for 30 to 120 minutes (preferably about 60 minutes) at room temperature (between 18 °C and 25 °C) so that the third HRP-conjugated antibody specifically binds to the second labelable human p-NFH antibody, removing the revealing solution and washing the wells 2 to 4 times (preferably 3 times) with a washing solution in order to remove the unbound third Labelled antibody from the plate;
Measure step 3) comprising:
   3a) adding a chromogenic HRP substrate to the wells,
   3b) incubating the plate protected from light for 5 to 30 minutes (preferably about 15 minutes) at room temperature (between 18 °C and 25 °C) so that the HRP of the third HRP-conjugated antibody converts the chromogenic HRP substrate into a coloured compound,
   3c) stopping the colour development with a stopping solution, and
   3d) measuring within 5 minutes the absorbance of each well using a microplate reader set to 450 nm, preferably with a reference wavelength set to 630 nm (acceptable range: 550-650 nm), and subtracting readings at 630 nm (550-650 nm) from the readings at 450 nm; and
Calculation step 4) comprising: calculating the concentration of pNFH in the CSF sample from a regression function of the standard curve obtained from wells comprising cascading dilutions of a control sample comprising p-NFH at a known concentration followed by multiplication by the dilution factor of the CSF sample.

Most preferably, the sandwich ELISA assay used for measuring the pNFH concentration in the CSF sample of the subject to be treated is performed using the Biovendor Human Phosphorylated Neurofilament H ELISA kit (BioVendor, cat #RD191138300R) used in MIROCALS clinical trial and following the manufacturer's instructions.

However, another kit might be used and a possibly distinct threshold value between high and medium CSF pNFH concentration determined by establishing a correlation function between the values obtained using the two distinct kits based on a calibration curve obtained with standard samples of known pNFH concentrations in the range observed in CSF samples of ALS patients.

Other Assays for Measuring the Concentration of p-NFH in CSF

While sandwich ELISA has been used in the MIROCALS clinical trial, any other suitable assay may be used for measuring the concentration of p-NFH in CSF in the context of the invention, provided that concentrations measured using the assay are correlated to those of a sandwich ELISA assay. Here also, a possibly distinct threshold value between high and medium CSF pNFH concentration can be determine by establishing a correlation function between the values obtained using the two distinct kits based on a calibration curve obtained with standard samples of known pNFH concentrations in the range observed in CSF samples of ALS patients.

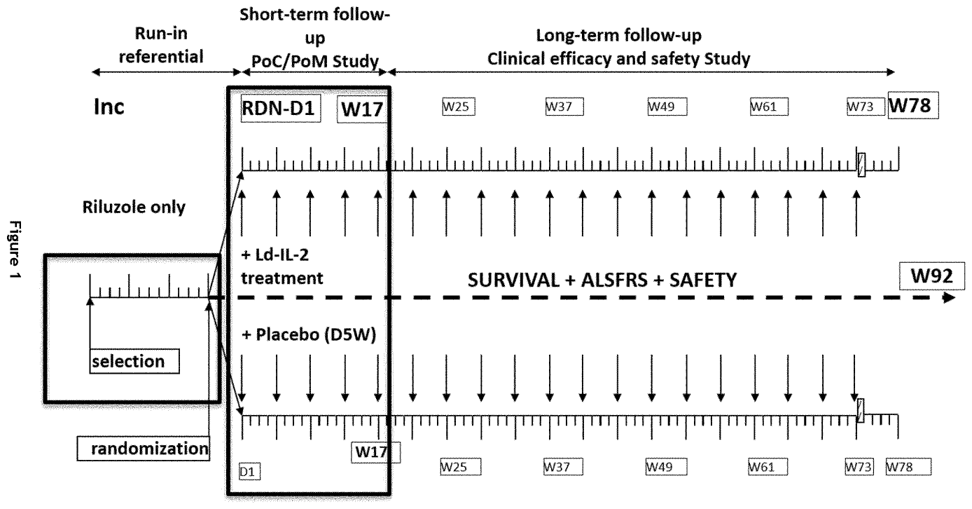

In particular, two other assays known in the art that may be used instead of sandwich ELISA include SIMOA® and electroluminescence (ECL) assays, as they are known to be more sensitive and their results are also known to be strongly linearly correlated to those obtained by ELISA (see Kuhle J. et al., Clin Chem Lab Med. 2016; 54:1655-1661, in particular Table 1 and FIGS. 1D, 1E and 1F). Another assay that may be used is Proximity Extension Assay (PEA).

“SIMOA®” or “Single molecule array” refers to a digital form of ELISA. SIMOA® is an assay based on the isolation of single immunocomplexes on paramagnetic beads using standard ELISA reagents. Briefly, paramagnetic particles coupled with the first anti-human p-NFH antibody recognizing a first epitope on human p-NFH as disclosed above are added to the sample. Then, a second anti-human p-NFH antibody recognizing a second epitope on human p-NFH as disclosed above is added. At low concentration, each bead will contain one bound pNFH or none. The sample is then loaded into arrays, in the SIMOA® disk containing more than 200 000 microwells, each large enough to hold one bead. The assay finishes by enzymatic signal amplification with substrate (generally fluorescent), imaging and data reduction. The detection at single molecule-level significantly improves analytical sensitivity. SIMOA® was employed in the study of NFs for the first time in 2015 (Gisslen et al., EBioMedicine. 2016; 3:135-140). Commercial SIMOA® has a sensitivity of 6-8 pg/mL; laboratory developed SIMOA® yielded detection thresholds lower than 1 pg/mL. When compared with ELISA this technology demonstrated a lower detection threshold for both NfL and pNfH. Moreover, SIMOA® is automated, assuring a good repeatability of results. A recent study, which tested pNfH on serum of ALS patients using both Simoa and ELISA, observed a lesser inter-assay variability with Simoa (Benatar et al., Neurology. 2020; 95:e59-69).

Except for the paramagnetic particles (which can be obtained commercially from Quanterix™), SIMOA® is based on standard ELISA reagents, and the same reagents as those mentioned above for the sandwich ELISA detection may thus be used.

“ECL” or “electroluminescence” assays use multiwell microplates (generally 96-well of 384-well) having electrodes (generally carbon electrodes) on which a capture antibody (for measuring the concentration of pNFH in CSF sample, the first anti-human p-NFH antibody recognizing a first epitope on human p-NFH as disclosed above) is grafted. The sample is then put in the microwells and captured by the capture antibody, and the microplate is incubated and washed. A detection antibody (for measuring the concentration of pNFH in CSF sample, the second anti-human p-NFH antibody recognizing a second epitope on human p-NFH as disclosed above) is added, and the microplate is incubated and washed. The detection antibody is labelled or labelable by an electrochemiluminescent molecule (generally SULFO-TAG labels), i.e. a molecule that when electrically stimulated undergoes electron-transfer reactions to form excited states that emit light. Electricity is then applied to the microplate electrodes leading to light emission by the electrochemiluminescent molecule. Light intensity is then measured to quantify analytes in the sample. Electrochemiluminescence (ECL) sensors are thus a combination of electrochemistry and measurement of visual luminescence. ECL microplates that can be coated with anti-human pNFH capture antibody can be obtained for instance from Meso Scale Diagnostics (MSD).

Electrochemiluminescence (ECL) assays have been used to study NFL in CSF (Gaiottino et al., PLoS One. 2013; 8:e75091; Gille et al., Neuropathol Appl Neurobiol. 2019; 45:291-304). Their analytical sensitivity is reported to be superior to ELISA (Kuhle et al., Clin Chem Lab Med. 2016; 54:1655-1661). Similar protocol may be used for measuring CSF pNFH concentration using antibodies specifically binding to pNFH instead of antibodies specifically binding to NFL.

“PEA” or “Proximity Extension Assay” refers to a dual-recognition immunoassay, where two matched antibodies labelled with unique DNA oligonucleotides simultaneously bind to a target protein in solution. This brings the two antibodies into proximity, allowing their DNA oligonucleotides to hybridize, serving as template for a DNA polymerase-dependent extension step. This creates a double-stranded DNA “barcode” which is unique for the specific antigen and quantitatively proportional to the initial concentration of target protein. The hybridization and extension are immediately followed by PCR amplification. The resulting DNA amplicon can then be quantified by quantitative real-time PCR (qPCR) or NGS. PEA reagents may notably be obtained from Olink.

### Assays for Measuring the Concentration of NFL or NFM in CSF, Blood, Serum or Plasma

Concentration of pNFH in CSF is preferred because pNFH, due to its phosphorylated status, is more resistant to proteases and thus to sample treatment variability. In addition, blood, serum or plasma pNFH is too low to permit reliable measure.

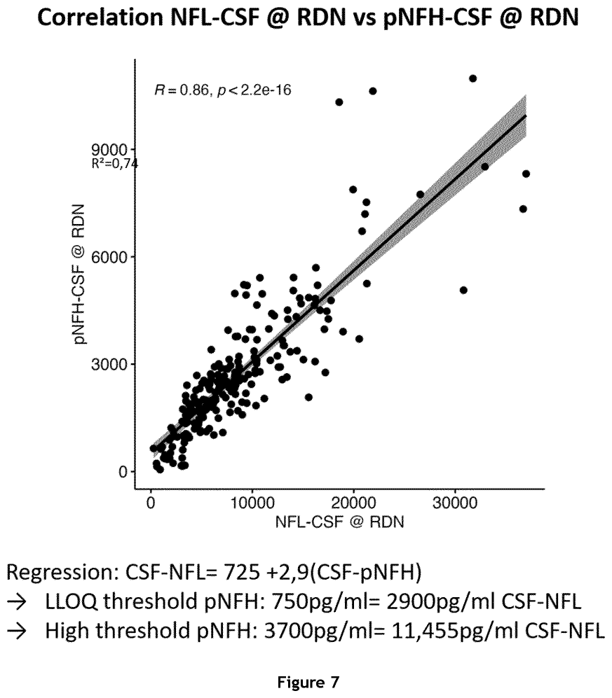

However, concentration of NFL or NFM in CSF, blood, serum or plasma may still be used in the uses and methods according to the invention, as blood, serum or plasma NFL concentration is linearly correlated to CSF NFL concentration when measured using SIMOA® or ECL assays (see Kuhle J. et al., Clin Chem Lab Med. 2016; 54:1655-1661, in particular Table 1 and FIGS. 1B and 1C). Moreover, CSF pNFH concentration is expected to be linearly correlated to CSF NFL or NFM concentrations, as neurofilament use a stoechiometric proportion of the light, medium and high chains. This correlation is further illustrated in Example 1 below, where it is clearly shown that CSF pNFH concentration is linearly correlated to CSF NFL concentration, even when using two distinct methods (sandwich ELISA for pNFH and SIMOA® for NFL, see FIG. 7).

Assays for measuring the concentration of NFL or NFM in CSF, blood, serum or plasma are the same as those disclosed above for measuring the concentration of pNFH in CSF, except that antibodies specifically binding to NFL or NFM should be used in place of those specifically binding to pNFH.

In addition, kits for measuring the concentration of NFL in CSF, blood, serum or plasma are commercially available. For instance, the commercially available ELISA UmanDiagnostics NF-Light® assay may be used for measuring the concentration of NFL in CSF or blood, serum or plasma. This kit uses two highly specific, non-competing monoclonal antibodies (47:3 as capture antibody, see; and 2:1 as detection antibody, see Norgren N et al. (2003) Brain Res 987: 25-31 and Norgren N. et al. (2002) Hybrid Hybridomics 21: 53-59) to quantify soluble NFL in CSF, blood, serum or plasma samples. The antibodies of this kit may also be used in sandwich SIMOA®, ECL, or PEA assays. And other non-competing anti-human NFL antibodies may be used in SIMOA®, ECL, or PEA assays, or any other suitable assay as defined above.

The concentration of NFM is less currently analyzed. However, the same protocols as for pNFH or NFL may be used using two non-competing anti-human NFM antibodies instead of two non-competing anti-human pNFH antibodies or two non-competing anti-human NFL antibodies. Anti-human NFM antibodies are commercially available, for instance from Aveslabs and MYBioSource (catalog numbers: MBS607436, MBS620458, MBS612396, MBS625130, MBS626981, MBS605999, MBS9608256, MBS421706). In addition, a Human Neurofilament medium polypeptide ELISA Kit (catalog number: MBS9428478) is available from MYBioSource.

### Threshold Values

Threshold values for the concentration of p-NFH measured in CSF by sandwich ELISA In the MIROCALS clinical trial, the inventors have found that low dose IL-2 treatment is particularly efficient for treating ALS in subjects who have a low or medium (lower than 3700 pg/ml with the specifically used sandwich ELISA assay) concentration of p-NFH in cerebrospinal fluid (CSF p-NFH) before human IL-2 administration when measured by a specific sandwich enzyme-linked immunosorbent assay (ELISA).

Therefore, in an embodiment of the invention, the treated human subject has a low or a medium concentration of p-NFH in cerebrospinal fluid (CSF p-NFH) before human IL-2 administration when measured by a sandwich ELISA assay. Preferably, human IL-2 is administered only to human subjects with ALS who have been selected for low or medium CSF p-NFH concentration based on a CSF sample (i.e. CSF pNFH has been measured in a CSF sample taken before human IL-2 by a sandwich ELISA assay and found to be low or medium, i.e. lower than a CSF_p-NFH_medium-high_sandwich-ELISA threshold value). As for any other concentration range, the concentrations of p-NFH in CSF samples from a cohort of ALS patients may be categorized as low, medium and high based on the mean concentration of p-NFH in CSF samples from a cohort of ALS patients similar to the cohort used in MIROCALS, i.e. a cohort comprising De novo patients, Possible, Probable, or Laboratory-Supported Probable, or Definite ALS by El Escorial Revised ALS diagnostic criteria, with a median disease duration or 8-12 months (preferably about 10 months), a median time from diagnosis of less than 2 months (preferably less than 1.5 months), and a repartition of El Escorial Category comprising about 8-14% (preferably about 11%) Possible versus about 20-30% (preferably about 24%) Definite ALS. A “low concentration” refers to a concentration lower than about one third of the mean concentration of p-NFH in CSF samples from the above defined cohort of ALS patients, which may correspond to the lower limit of quantification (also referred to as LLOQ) of the sandwich ELISA. A “medium concentration” refers to a concentration between about one third (or LLOQ of the sandwich ELISA) and about 1.6 times the mean concentration of p-NFH in CSF samples from the cohort of ALS patients. And a “high concentration” refers to a concentration higher than about 1.6 times the mean concentration of p-NFH in CSF samples from the cohort of ALS patients.

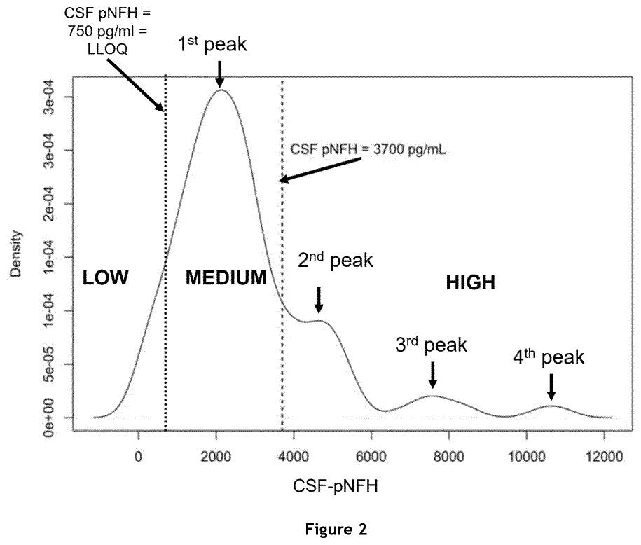

Alternatively, the concentrations of p-NFH in CSF samples from a cohort of ALS patients may be categorized as low, medium and high based on the analysis of the distribution of CSF pNFH concentrations of a cohort of ALS patients similar to the cohort used in MIROCALS, i.e. a cohort comprising De novo patients, Possible, Probable, or Laboratory-Supported Probable, or Definite ALS by El Escorial Revised ALS diagnostic criteria, with a median disease duration or 8-12 months (preferably about 10 months), a median time from diagnosis of less than 2 months (preferably less than 1.5 months), and a repartition of El Escorial Category comprising about 8-14% (preferably about 11%) Possible versus about 20-30% (preferably about 24%) Definite ALS. A graph representing the density of patients depending on their p-NFH concentration in CSF is constructed, which should present a multimodal distribution comprising, when the p-NFH concentration in CSF increases, a first highest peak of density followed by one or more (preferably two or three) lower peaks of density (see FIG. 2). Based on the multimodal distribution curve, a threshold value separating High CSF-pNFH concentrations from Medium and Low CSF-pNFH concentrations may be selected between the first and second density peaks. In this embodiment, the threshold value separating Medium CSF-pNFH concentrations from Low CSF-pNFH concentrations is preferably the CSF pNFH concentration corresponding to the lower limit of quantification (LLOQ) of the assay used to measure CSF pNFH, i.e. the lower limit at which the assay can provide quantitative results.

Although the precise concentration determined from a CSF sample may slightly vary depending on the specific sandwich ELISA assay used, based on the cohort of ALS patients from the MIROCALS clinical trial, low, medium and high concentration of CSF p-NFH before human IL-2 administration when measured by a sandwich ELISA assay may be defined as:
Low concentration: a concentration lower than a first threshold value comprised between 500 and 1000 pg/ml, preferably comprised between 600 and 900 pg/ml, between 700 and 800 pg/ml, more preferably the first threshold value is about 750 pg/ml;
Medium concentration: a concentration between a first threshold value comprised between 500 and 1000 pg/ml and a second threshold value comprised between 3000 and 4500 pg/ml, preferably the first threshold value is comprised between 550 and 950 pg/ml and the second threshold value is comprised between 3200 and 4200 pg/ml; the first threshold value is comprised between 600 and 900 pg/ml and the second threshold value is comprised between 3400 and 4000 pg/ml; the first threshold value is comprised between 650 and 850 pg/ml and the second threshold value is comprised between 3600 and 3800 pg/ml; more preferably the first threshold value is about 750 pg/ml and the second threshold value is about 3700 pg/ml;
High concentration: a concentration higher than a second threshold value comprised between 3000 and 4500 pg/ml, preferably comprised between 3200 and 4200 pg/ml, comprised between 3400 and 4000 pg/ml, comprised between 3600 and 3800 pg/ml, more preferably the second threshold value is about 3700 pg/ml.

As the treated subject has or has been selected based on a measure performed in vitro in a sample obtained before human IL-2 administration for a low to medium concentration of CSF p-NFH before human IL-2 administration when measured by a sandwich ELISA assay, he/she preferably has or has been selected based on a measure performed in vitro in a sample obtained before human IL-2 administration for a concentration of CSF p-NFH before human IL-2 administration when measured by the sandwich ELISA assay lower than 4500 pg/ml, preferably lower than 4400 pg/ml, lower than 4300 pg/ml, lower than 4200 pg/ml, lower than 4100 pg/ml, more preferably lower than 4000 pg/ml, lower than 3900 pg/ml, lower than 3800 pg/ml, and most preferably lower than 3700 pg/ml, when measured by the sandwich ELISA assay.

The inventors also found out that subject with a low concentration of CSF p-NFH before human IL-2 administration when measured by the sandwich ELISA assay could survive without the low dose IL-2 treatment (at least during the observation window of MIROCALS, i.e. only 92 weeks), so that the treatment is preferably administered to subjects with a medium concentration of CSF p-NFH before human IL-2 administration when measured by the sandwich ELISA assay (any embodiment disclosed above), as these subjects are those most benefitting from the treatment.

Subjects with a medium concentration of CSF p-NFH before human IL-2 administration when measured by the sandwich ELISA assay preferably have or have been selected based on a measure performed in vitro in a sample obtained before human IL-2 administration for a concentration of CSF p-NFH before human IL-2 administration when measured by the sandwich ELISA assay that is:
between about one third of and less than about 1.6 times the mean concentration of p-NFH in CSF samples from a cohort of ALS patients as defined above; or
between about 500 and 4500 pg/ml, preferably between about 550 and 4200 pg/ml, between about 600 and 4000 pg/ml, between about 700 and 3800 pg/ml, more preferably between 750 and 3700 pg/ml.

However, the treatment may also preferably be administered to subjects with a low or medium concentration of CSF p-NFH before human IL-2 administration when measured by the sandwich ELISA assay, as low dose IL-2 treatment as defined herein is safe and well tolerated and may probably still have advantageous long-term effects in subjects with a low concentration of CSF p-NFH before human IL-2 administration when measured by the sandwich ELISA assay.

Threshold Values for the Concentration of p-NFH Measured in CSF by Other Assays

As illustrated in Table 1 and FIGS. 1C, 1D and 1E of Kuhle J. et al., Clin Chem Lab Med. 2016; 54:1655-1661, while the values measured for the concentrations of NFL measured in CSF by sandwich ELISA, SIMOA® and ECL differ due to distinct sensitivities of the methods, the values obtained by one assay are strongly correlated to the values obtained by another assay. In addition, CSF pNFH concentration is expected to be linearly correlated to CSF NFL concentration, as neurofilament use a stoechiometric proportion of the light, medium and high chains, which is further demonstrated in Example 1 below (see FIG. 7) and the inventors have demonstrated that CSF-NFL concentration at randomization is also predictive of low dose IL-2 treatment efficiency (see Table 11). It follows that the values of CSF pNFH concentrations obtained by sandwich ELISA are linearly correlated to the values of CSF pNFH concentrations obtained by other assays, in particular by SIMOA® or ECL assays. Correlation with PEA assay is also expected.

As a result, based on threshold values disclosed herein for CSF pNFH concentrations obtained using sandwich ELISA, a skilled person could easily obtain corresponding threshold values for another assay (in particular SIMOA®, ECL or PEA assays, notably SIMOA® or ECL assays) by routine experiments. For instance, the samples of a cohort of ALS patient may be treated by the two assays and a regression determined. Alternatively, a control sample containing a known amount of pNFH could be used for measuring cascading dilutions of this control sample by the two assays and determination of a regression.

Moreover, values obtained by various assays are not only linearly correlated, but also close to each other. As a result, although the correlated thresholds that may be determined for another assay measuring CSF pNFH concentration may be slightly different from those of a sandwich ELISA, the absolute values or ranges of values of CSF pNFH as assessed by sandwich ELISA defined in the previous section may also be used for other assays used for measuring CSF pNFH, in particular SIMOA®, ECL or PEA assays, notably SIMOA® or ECL assays.

### Threshold Values for the Concentration of NFL or NFM in CSF, Blood, Serum or Plasma

As explained above, linear correlations exist between the concentrations of the different types of neurofilaments (NFL, NFM and pNFH), between CSF and blood/serum/plasma samples, and between values obtained using different assays.

Therefore, based on threshold values disclosed herein for CSF pNFH concentrations obtained using sandwich ELISA, a skilled person could easily obtain by routine experiments corresponding threshold values for concentrations of NFL or NFM in CSF, blood, serum or plasma in any assay of interest, in particular sandwich ELISA, SIMOA®, ECL or PEA assays, notably ELISA, SIMOA® or ECL assays.

In particular, CSF NFL concentrations have been determined by SIMOA® in MIROCALS, and suitable thresholds have been defined based on regression analysis of the correlation between CSF pNFH measured by sandwich ELISA and CSF NFL measured by SIMOA® (see Example below and FIG. 7).

The low, medium and high concentrations of CSF NFL may thus be defined as:
Low concentration: a concentration lower than a first threshold value comprised between 2200 and 3600 pg/ml, preferably comprised between 2400 and 3400 pg/ml, comprised between 2600 and 3200 pg/ml, preferably comprised between 2800 and 3000 pg/ml, about 2900 pg/ml;
Medium concentration: a concentration between a first threshold value comprised between 2200 and 3600 pg/ml and a second threshold value comprised between 9400 and 13800 pg/ml, preferably the first threshold value is comprised between 2400 and 3400 pg/ml and the second threshold value is comprised between 10000 and 12900 pg/ml; the first threshold value is comprised between 2600 and 3200 pg/ml and the second threshold value is comprised between 10600 and 12400 pg/ml; the first threshold value is comprised between 2800 and 3000 pg/ml and the second threshold value is comprised between 11200 and 11800 pg/ml; more preferably the first threshold value is about 2900 pg/ml and the second threshold value is about 11455 pg/ml;
High concentration: a concentration higher than a second threshold value comprised between 9400 and 13800 pg/ml, preferably comprised between 10000 and 12900 pg/ml, comprised between 10600 and 12400 pg/ml, comprised between 11200 and 11800 pg/ml, more preferably the second threshold value is about 11455 pg/ml.

In an embodiment, the subject thus preferably has or has been selected based on a measure performed in vitro in a sample obtained before human IL-2 administration for a concentration of NFL in cerebrospinal fluid (CSF NFL) before human IL-2 administration lower than 13800 pg/ml, preferably lower than 12900 pg/ml, more preferably lower than 12400 pg/ml, lower than 11800 pg/ml, more preferably lower than 11455 pg/ml, when measured by any suitable assay as defined above, and in particular when measured by a Single molecule array (SIMOA®) assay.

When only the medium concentration is targeted, the subject preferably has or has been selected based on a measure performed in vitro in a sample obtained before human IL-2 administration for a concentration of NFL in cerebrospinal fluid (CSF NFL) before human IL-2 administration between 2200 and 13800 pg/ml, preferably between 2400 and 12900 pg/ml, between 2600 and 12400 pg/ml, between 2800 and 11800 pg/ml, more preferably between 2900 and 11455 pg/ml, when measured by any suitable assay as defined above, and in particular when measured by a Single molecule array (SIMOA®) assay.

### HUMAN IL-2

The claimed treatment relies on the administration of low doses of human IL-2 to the human ALS subject.

In the present description, “human interleukin 2” or “human IL-2” designates any source of human IL-2, including native human IL-2 or human IL-2 obtained by recombinant or synthetic techniques, including recombinant IL-2 polypeptides produced by microbial hosts. Nucleotide and amino acid sequences of native human IL-2 are disclosed, for instance, in the description of human IL-2 gene in Pubmed Entrez Gene reference 3558. A reference sequence for native human IL-2 protein may be found in NCBI Reference Sequence NP_000577.2 (version updated on Jan. 5, 2020), while a reference sequence for native human IL-2 mRNA may be found in NCBI Reference Sequence NM_000586.4 (version updated on Jan. 5, 2020).

Human IL-2 may consist of or comprise the native human IL-2 polypeptide sequence, or can be an active variant of the native human IL-2 polypeptide. Preferably recombinant human IL-2 is used, particularly recombinant human IL-2 produced by microbial hosts. Active variants of IL-2 have been disclosed in the literature. Variants of the native IL-2 can be fragments, analogues, and derivatives thereof. By “fragment” is intended a polypeptide comprising only a part of the intact polypeptide sequence. An “analogue” designates a polypeptide comprising the native polypeptide sequence with one or more amino acid substitutions, insertions, or deletions. Muteins and pseudopeptides are specific examples of analogues. “Derivatives” include any modified native IL-2 polypeptide or fragment or analogue thereof, such as glycosylated, phosphorylated, fused to another polypeptide or molecule, polymerized, etc., or through chemical or enzymatic modification or addition to improve the properties of IL-2 (e.g., stability, specificity, etc.). Active variants of native human IL-2 polypeptide generally have at least 75%, preferably at least 85%, more preferably at least 90% amino acid sequence identity to the amino acid sequence of native human IL-2 polypeptide. Methods for determining whether a variant IL-2 polypeptide is active are available in the art, examples of IL-2 variants being disclosed, for instance, in EP109748, EP136489, U.S. Pat. No. 4,752,585; EP200280, or EP118617, which are herein incorporated by reference. An active variant is, most preferably, a variant that activates Tregs.

Preferably, recombinant human IL-2, i.e., human IL-2 that has been prepared by recombinant DNA techniques, is used. The host organism used to express a recombinant DNA encoding human IL-2 may be prokaryotic (a bacterium such as E. coli) or eukaryotic (e.g., a yeast, fungus, plant or mammalian cell). Processes for producing recombinant IL-2 have been described e.g., in U.S. Pat. Nos. 4,656,132; 4,748,234; 4,530,787; or U.S. Pat. No. 4,748,234, which are herein incorporated by reference.

Human IL-2 for use in the present invention shall be in in pharmaceutically acceptable form, and notably in essentially pure form, e.g., at a purity of 95% or more, further preferably 96, 97, 98 or 99% pure.

Human IL-2 is commercially available, including for pharmaceutical uses, and has been authorized for use in human subjects. For instance, aldesleukin (trademark name Proleukin®) is an analog of the human interleukin-2 gene produced by recombinant DNA technology using a genetically engineered E. coli strain, which has been approved by the FDA in the treatment of cancers. Aldesleukin differs from native human interleukin-2 in the following ways:
a) aldesleukin is not glycosylated because it is derived from E. coli;
b) the molecule has no N-terminal alanine; the codon for this amino acid was deleted during the genetic engineering procedure;
c) the molecule has serine substituted for cysteine at amino acid position 125.

Aldesleukin will preferably be used in the invention.

However, other recombinant human IL-2 are available, such as:
Roncoleukin®, a medicinal form of recombinant human IL-2, isolated and purified from cells of the yeast Saccharomyces cerevisiae containing the gene of human IL-2;
Interking, a recombinant IL-2 with a serine at residue 125, sold by Shenzhen Neptunus;
Albuleukin, a recombinant human interleukin-2 (rIL-2) genetically fused to recombinant human serum albumin (rHSA).

### Human IL-2 Dose and Administration Schemes and Routes

As explained above, human Tregs constitutively express high levels of CD25, forming a high-affinity receptor for IL-2 absent on resting Teffs, and thus respond to low concentrations of IL-2, insufficient to stimulate Teffs. Since the claimed treatment is intended to expand the number and function of Tregs but not to expand or stimulate Teffs, each dose of human IL-2 administered to the ALS subject is kept low, between 0.1×10⁶ to 3×10⁶ international units (IU). In addition, in the context of type 1 diabetes, it has been shown that doses up to 3×10⁶ IU were safe, although more non-serious adverse events occurred at the highest dose of 3×10⁶ IU (Hartemann A et al. Lancet Diabetes Endocrinol 2013; 1: 295-305). Each administered human IL-2 dose should also be kept to at most 3×10⁶ IU in order to limit possible toxicity.

As explained above, Tregs are exclusively reliant on IL-2 for their generation, activation and survival (Malek T R, Bayer A L. Nat Rev Immunol 2004; 4: 665-74). Moreover, the half-like of aldesleukin administered to human patients is typically about 2-3 hours (see e.g. Proleukin® label). Therefore, in order to obtain sustained expansion of Treg numbers and immunosuppressive function, human IL-2 is typically administered repeatedly to the ALS subjects.

In a preferred embodiment, human IL-2 is administered as repeated, preferably sub-cutaneous, injections of 0.1×10⁶ to 3×10⁶ IU of human IL-2, preferably 0.2×10⁶ to 3×10⁶ IU of human IL-2, 0.3×10⁶ to 3×10⁶ IU of human IL-2, 0.4×10⁶ to 3×10⁶ IU of human IL-2, more preferably 0.5×10⁶ to 3×10⁶ IU of human IL-2, 0.6×10⁶ to 3×10⁶ IU of human IL-2, 0.7×10⁶ to 3×10⁶ IU of human IL-2, 0.8×10⁶ to 3×10⁶ IU of human IL-2, 0.9×10⁶ to 3×10⁶ IU of human IL-2, 1×10⁶ to 3×10⁶ IU of human IL-2, 1×10⁶ to 2×10⁶ IU of human IL-2, in particular 0.5×10⁶ IU of human IL-2, 1×10⁶ IU of human IL-2, or 2×10⁶ IU of human IL-2. Injections may be performed according to various administration schemes, including:
Administrating schemes based on repeated cycles of once-daily or several daily injections of human IL-2, separated by periods without human IL-2 injections; and
Continuous metronomic administration, i.e. once-daily or several daily injections of human IL-2.

In the context of IMODALS clinical trial (see Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044), it has been shown that, contrary to what Thonhoff et al had suggested (Thonhoff J R et al. Neurol Neuroimmunol Neuroinflammation 2018; 5: e465), a first 5 days cycle starting at D1 (cycle 1) of 1×10⁶ or 2×10⁶ IU/day human IL-2 administered once daily subcutaneously was sufficient to significantly increase Tregs numbers and function up to D29. Moreover, further identical 5 days cycles at week 5 (cycle 2, starting on D29) and week 9 (cycle 3, starting on D57) have been administered, and the peak of Tregs numbers and frequencies during cycle 3 was higher than that observed during cycle 1, suggesting that successive treatment cycles have residual effects that might be cumulative. This suggestion is further supported by significantly higher iAUC trough T_{reg} levels (measuring the residual Treg change before beginning a new cycle) in IL2 arms as compared to placebo.

Therefore, a cycle of 3 to 7 consecutive days of once-daily sub-cutaneous injection of 0.1×10⁶ to 3×10⁶ IU of human IL-2, preferably 0.2×10⁶ to 3×10⁶ IU of human IL-2, 0.3×10⁶ to 3×10⁶ IU of human IL-2, 0.4×10⁶ to 3×10⁶ IU of human IL-2, more preferably 0.5×10⁶ to 3×10⁶ IU of human IL-2, 0.6×10⁶ to 3×10⁶ IU of human IL-2, 0.7×10⁶ to 3×10⁶ IU of human IL-2, 0.8×10⁶ to 3×10⁶ IU of human IL-2, 0.9×10⁶ to 3×10⁶ IU of human IL-2, 1×10⁶ to 3×10⁶ IU human IL-2, 0.5×10⁶ to 2×10⁶ IU of human IL-2, 1×10⁶ to 2×10⁶ IU of human IL-2, in particular 0.5×10⁶ IU of human IL-2, 1×10⁶ IU of human IL-2, or 2×10⁶ IU of human IL-2, is expected to significantly increase Tregs numbers during the cycle and at least up to 3 further weeks.

In the therapeutic uses or methods according to the invention, several cycles of 3 to 7 consecutive days of once-daily sub-cutaneous injection of 0.1×10⁶ to 3×10⁶ IU of human IL-2, preferably 0.2×10⁶ to 3×10⁶ IU of human IL-2, 0.3×10⁶ to 3×10⁶ IU of human IL-2, 0.4×10⁶ to 3×10⁶ IU of human IL-2, more preferably 0.5×10⁶ to 3×10⁶ IU of human IL-2, 0.6×10⁶ to 3×10⁶ IU of human IL-2, 0.7×10⁶ to 3×10⁶ IU of human IL-2, 0.8×10⁶ to 3×10⁶ IU of human IL-2, 0.9×10⁶ to 3×10⁶ IU of human IL-2, 1×10⁶ to 3×10⁶ IU human IL-2, 0.5×10⁶ to 2×10⁶ IU of human IL-2, 1×10⁶ to 2×10⁶ IU of human IL-2, in particular 0.5×10⁶ IU of human IL-2, 1×10⁶ IU of human IL-2, or 2×10⁶ IU of human IL-2, are thus preferably administered to the subject. More preferably, each cycle consists of 5 consecutive days of once-daily sub-cutaneous injection of 0.1×10⁶ to 3×10⁶ IU of human IL-2, preferably 0.2×10⁶ to 3×10⁶ IU of human IL-2, 0.3×10⁶ to 3×10⁶ IU of human IL-2, 0.4×10⁶ to 3×10⁶ IU of human IL-2, more preferably 0.5×10⁶ to 3×10⁶ IU of human IL-2, 0.6×10⁶ to 3×10⁶ IU of human IL-2, 0.7×10⁶ to 3×10⁶ IU of human IL-2, 0.8×10⁶ to 3×10⁶ IU of human IL-2, 0.9×10⁶ to 3×10⁶ IU of human IL-2, 0.5×10⁶ to 2×10⁶ IU of human IL-2, 1×10⁶ to 2×10⁶ IU of human IL-2, in particular 0.5×10⁶ IU of human IL-2, 1×10⁶ to 3×10⁶ IU IL-2, or 2×10⁶ IU human IL-2.

Since each cycle is expected to significantly increase Tregs numbers and function during the cycle and at least about 3 further weeks, the cycles are preferably administered every 2 to 6 weeks, preferably every 2 to 5 weeks, more preferably every 2 to 4 weeks, in particular every 2, 3 or 4 weeks.

However, while the above administrations schemes are preferred, other administration schemes able to significantly increase Tregs numbers and function without unacceptable toxicity may be defined by those skilled in the art, based on their knowledge of low dose IL-2 administration in humans in other contexts.

Indeed, while there were teachings in the art suggesting that ALS subjects had dysfunctional Tregs (Beers D R et al. JCI Insight. 2017; 2(5):e89530; Thonhoff J R et al. Curr Opin Neurol 2018; 31: 635-9) and did not respond to low dose IL-2 administration (Thonhoff J R et al. Neurol Neuroimmunol Neuroinflammation 2018; 5: e465), contrary to other human subjects, it has been shown in IMODALS that ALS subjects are in fact able to favorably respond to ld-IL-2, resulting in a significant increase in Tregs numbers and function, and foremost decrease in CCL2 plasma concentration, a marker of ALS disease activity (Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044). Based on this finding, knowledge of the effects of low dose IL-2 in other human subjects may be used to design other suitable administration schemes.

For instance, while subcutaneous administration has been used in IMODALS clinical trial, it has been shown in human cancer subjects that IL-2 administered intravenously resulted in significant increase in Tregs numbers (Ahmadzadeh M, Rosenberg SA. Blood 2006; 107:2409-14). Therefore, in the therapeutic uses and methods according to the invention, human IL-2 is preferably administered via subcutaneous or intravenous route.

Since subcutaneous is easier and better tolerated and shown to be efficient in the IMODALS clinical trial, subcutaneous route is nevertheless preferred.

With respect to the dose, while each single dose should not be higher than 3×10⁶ IU in order to limit possible toxicity, results obtained in IMODALS clinical trial showed that the effect of low dose human IL-2 on Tregs numbers and function in ALS subjects was dose-dependent, the highest effects being obtained with the highest dose of 2×10⁶ IU human IL-2 once daily during each of the 5 days cycles. Therefore, when using an administration scheme comprising repeated and separated cycles of low dose human IL-2 administration, during a cycle, a daily dose of 1×10⁶ IU to 2×10⁶ IU, preferably 2×10⁶ IU, is preferred. The daily dose may however be administered either in a single daily administration or in several separated lower doses. For instance, in order to reach a daily dose of 2×10⁶ IU, a single dose of 2×10⁶ IU may be administered once daily, or this daily dose of 2×10⁶ IU may be split into 2 or more lower doses, such as 2 doses of 1×10⁶ IU (for instance one in the morning and the other in the evening), 3 doses of 0.67×10⁶ IU (for instance one in the morning, one in the middle of the day and the 3^{rd} in the evening), or even 4 doses of 0.5×10⁶ IU. Such splitting of the daily dose may in particular be used in ALS subjects suffering from adverse events when administered a single daily dose of 2 to 3×10⁶ IU.

In addition, while the administration scheme used in IMODALS clinical trial is based on 5 days cycles every 4 weeks, other administration schemes may be contemplated. For instance, alternative administration schemes may be based on:
longer cycles with a longer period between 2 cycles;
shorter cycles with a shorter period between 2 cycles;
continuous administration of low dose human IL-2 (no cycles).

For instance, when using administration cycles, while each cycle may notably vary between 3 and 7 days, shorter (such as 2 days) or longer (such as 8, 9, 10, 11, 12, 13, 14 days, or even 3 or 4 weeks) cycles may be used. When using shorter cycles (such as 2 days), the cycles will preferably be repeated more often than the every 4 weeks schedule used in IMODALS, such as every 3 weeks, every 2 weeks, every 10 days, or every week. When using longer cycles (such as 8, 9, 10, 11, 12, 13, 14 days, or even 3 or 4 weeks), the cycles will preferably be repeated as often or a little bit less often than the every 4 weeks schedule used in IMODALS, such as every 4 weeks, every 5 weeks, or every 6 weeks. However, due to the relatively short duration of the effects of human IL-2, the period between cycles should not be too much increased.

Continuous (also referred to as “metronomic”) administration of low dose human IL-2 (no cycles) may also be contemplated. While administration schemes based on repeated cycles of low dose human IL-2 have been used in IMODALS and MIROCALS and in other autoimmune diseases based on previous knowledge derived from cancer therapy and for commodity for the subject, continuous administration of low dose human IL-2 may still be considered, in particular if a pump permitting continuous administration of low dose human IL-2 (similar to those used for delivering insulin to diabetic subjects) is used. In this case, similar daily doses may be contemplated. However, in view of the Treg tendency to accumulate after 3 cycles as observed in IMODALS (see WO2021/176044), lower cumulative daily doses of human IL-2 may be contemplated, such as a daily dose of 0.1×10⁶ to 2×10⁶ IU, preferably 0.1×10⁶ to 1.5×10⁶ IU, 0.1×10⁶ to 1×10⁶ IU, or even 0.1×10⁶ to 0.5×10⁶ IU. This type of treatment may notably be contemplated in ALS patient showing adverse effects to higher daily doses of human IL-2.

More generally, while each single dose should be comprised between 0.1×10⁶ and 3×10⁶ IU, the clinician will know how to adapt the administration scheme in order to observe efficiency without unacceptable toxicity. In particular, starting from a given administration scheme (such as one of the schemes selected in IMODALS and MIROCALS clinical trials), a clinician will be able to monitor the number or frequency of Tregs, their immunosuppressive function, and/or the serum, plasma or cerebrospinal fluid (CSF) concentration of CCL2 and/or CCL17 and/or CCL18, as well as possible adverse events, and adapt the administration scheme in order to optimize the benefit to risk ratio (improving efficiency based on the number or frequency of Tregs, their immunosuppressive function, and/or the serum, plasma or cerebrospinal fluid (CSF) concentration of CCL2 markers and/or CCL17 and/or CCL18, and/or limiting drug-related adverse events).

In this context, in an embodiment of the present invention, the treatment comprises:
a) measuring Tregs number or frequency (in blood), and/or Tregs immunosuppressive function, and/or serum or plasma or CSF concentrations of CCL2, and/or serum, plasma or CSF concentrations of CCL17 and/or CCL18, at baseline from a biological sample of the subject (i.e on the day of starting the low dose human IL-2 treatment),
b) administering human IL-2 to the subject according to a first administration scheme according to the invention comprising either repeated separated cycles of human IL-2 administration or continuous-metronomic-human IL-2 administration,
c) monitoring drug-related adverse events and measuring the same parameter(s) as at baseline from a biological sample of the subject taken 1-3 days following the end of a cycle of treatment or at least 7 days after continuous-metronomic-human IL-2 administration, and
d) continuing the first administration scheme when results are acceptable, or designing a second administration scheme depending on the results of step c).

In step d), the cycles or continuous daily dose may for instance be decreased or split into several lower single doses (instead of a once daily dose) if the ALS subject experiences poorly tolerated adverse events with compliance issues. If both the first and second administration schemes are based on cycles and the daily dose of human IL-2 during cycles is decreased, then the duration of each cycle may be increased to compensate. Alternatively, since lower daily doses are expected to be sufficient when using continuous administration rather than cycles, the second administration scheme may be based on continuous administration instead of cycles.

Conversely, if the ALS subject does not experience unacceptable toxicity but the therapeutic effect—as measured using Tregs number, frequency or immunosuppressive function (markers negatively correlated to disease progression) or CCL2, CCL17 and/or CCL18 plasma or CSF concentration (CCL2 is positively correlated to disease progression, CCL17 and/or CCL18 are indicative of a change of macrophage polarization from an pro-inflammatory M1 phenotype to an anti-inflammatory M2 phenotype)—is not sufficient, then the daily dose of cycles or continuous administration doses may be increased in order to increase chances that the ALS subject responds to the treatment, provided that each single dose administered to the subject is at most 3×10⁶ IU. Since Tregs number, frequency or immunosuppressive function, and CCL2 plasma or CSF concentration have more particularly been correlated to disease progression, at least one of these markers will preferably be used. Because plasma or serum CCL2 is easier to measure and more reliable, CCL2 plasma or serum concentration is even more preferred.

CCL2 concentrations can be measured in plasma, in serum or in CSF. The measurements can be performed on fresh or frozen (−20 °C) plasma or serum or CSF samples. CCL2 concentrations are measured in fresh or frozen plasma or serum or CSF using solid phase immune-assay such as enzyme-linked immunosorbent assay (ELISA) method (as done in MIROCALS study) or cytometric beads assay (as done in IMODALS). IL2 unit dose increase would be considered when CCL2 concentrations, are over 80% of pretreatment baseline concentrations (ie, showing less than 20% decrease on treatment).

Whatever the selected administration scheme (which may be modified during treatment, as explained above), the treatment is preferably administered for the life-time of the subject or until unacceptable drug-related Serious Adverse Event.

### Other Treatments Preferably not Administered to the Human Subject

In the therapeutic uses and methods of the invention, the treatment does preferably not comprise the administration of regulatory T cells to the subject. Indeed, contrary to what had been suggested by Thonhoff et al (Thonhoff J R et al. Neurol Neuroimmunol Neuroinflammation 2018; 5: e465), it was found in IMODALS that the mere injection of low dose human interleukin 2 (ldIL-2) is sufficient to induce a significant improvement not only in Treg numbers but also most importantly in their suppressive function in all ALS subjects, without the need for Treg isolation and ex vivo expansion prior to re-injection (Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044).

“Regulatory T cells” or “Tregs” are T lymphocytes having immunosuppressive activity. Natural Tregs are characterized by a CD4⁺CD25⁺Foxp3⁺ phenotype. Tregs are also characterized by their functional ability to inhibit proliferation of T-effector cells.

While human IL-2 may be administered to the ALS subject in combination with another treatment (see below), alternatively or in addition to the lack of combined treatment with Tregs, the claimed treatment is also preferably not combined with one or more of the following treatments:
In a first embodiment, in addition to the lack of combined treatment with Tregs, the human IL-2 administered to the subject is not complexed with anti-human IL-2 antibodies.
Complexes of IL-2 with anti-IL-2 antibodies, rather than purified IL-2, have been used by Sheean et al in transgenic SOD1 ALS mouse, because the IL-2/IL-2 monoclonal antibody complexes were considered to increase the biological activity of IL-2, and because the selected anti-IL-2 monoclonal antibody clone permitted to confer specificity to cells expressing high-affinity αβγ IL-2R (CD25hiCD4+Foxp3+ Tregs and activated effector T-cells) rather than those expressing low-affinity βy IL-2R (memory CD8+ cells or natural killer cells) (Sheean R K et al. JAMA Neurol. 2018; 75(6):681-689, see Methods, section “Data Collection From Animal Participants”, paragraph 3).
However, it was found in IMODALS that administration of low doses of non-complexed human IL-2 in ALS subjects is sufficient to specifically increase the number, proportion and suppressive function of Tregs (Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044).
As a result, in the context of the invention, human IL-2 administered to the subject is preferably not complexed with anti-human IL-2 antibodies.
In a second embodiment, in addition to the lack of combined treatment with Tregs, the claimed treatment is also preferably not combined with rapamycin or any other agent suppressive of effector T-cells (Teffs).
In the transgenic SOD1 ALS mouse, Sheean et al not only used IL-2 complexed with anti-IL-2 antibodies, but also combined complexed IL-2 with rapamycin treatment. Rapamycin is an immunosuppressant drug known to particularly suppress effector T-cells (Teffs) expansion, and has been used by Sheean et al. in combination with IL-2/anti-IL-2 complexes in order to specifically expand Treg cells with an activated phenotype and to exert immunosuppressive function, the combination being considered as essential because it inhibits proliferation of T effector cells, enabling selective expansion of Tregs (Sheean R K et al. JAMA Neurol. 2018; 75(6):681-689, see Methods, section “Data Collection From Animal Participants”, paragraph 3).
However, it was also found in IMODALS that mere administration of low doses of non-complexed human IL-2 in ALS subjects, without the combined administration of an agent immunosuppressive of Teffs such as rapamycin, is sufficient to specifically increase the number, proportion and function of Tregs (Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844; WO2021/176044).
As a result, in the context of the invention, in addition to the lack of combined treatment with Tregs, the claimed treatment is also preferably not combined with rapamycin or any other agent suppressive of effector T-cells (Teffs).

“Effector T cells” or “Teffs” include all CD4 cells other than Tregs. In particular, Teffs do not constitutively express FOXP3.
In a preferred embodiment, in addition to the lack of combined treatment with Tregs, the human IL-2 administered to the subject is not complexed with anti-human IL-2 antibodies and the claimed treatment is also not combined with rapamycin or any other agent suppressive of Teffs (as disclosed above).
   Other Treatments that May be Administered to the Human Subject

Currently, the only treatment available for the treatment of ALS is riluzole (2-Amino-6-(trifluoromethoxy)benzothiazole, CAS number 1744-22-5, trademark name Riluteko), a compound of formula:

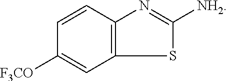 [SMILES: Nc1nc2ccc(OC(F)(F)F)cc2s1]

Approved for the treatment of ALS by the FDA in 1995, riluzole has been shown to be associated with a short median survival benefit of 2-3 months equating to a 9% absolute increase in 1-year survival (Miller R G, et al. Cochrane Database of Systematic Reviews 2012, Issue 3. Art. No.: CD001447).

While this short survival benefit is not satisfying, it is still better than no treatment and most ALS subjects are thus treated by riluzole.

Therefore, the treatment of the invention with low dose human IL-2 preferably further comprises administering riluzole to said subject.

In ALS, riluzole is typically used at a daily dose of 100 mg/day by oral route, taken in two equal doses of 50 mg separated by about 12 hours. In case of toxicity, a lower daily dose, such as a daily dose of 50 mg/day by oral route, taken in two equal doses of 25 mg separated by about 12 hours, may be used.

When the treatment of the invention is combined with riluzole treatment, riluzole is thus preferably orally administered at a daily dose of 50 mg to 100 mg, taken in two equal doses of 25 mg to 50 mg separated by about 12 hours.

Further optional treatments commonly administered to ALS subjects may further be administered to the ALS subjects in the context of the invention, including antidepressants (when the ALS subject suffers from depressive symptoms), analgesics (to limit pain), anticholinergics (in case of hypersiallorhea), and antibiotics (in case of bacterial infection), or any other treatment found to be useful in ALS.

### Medical Use and Methods of Treatment in all ALS Patients

The present invention also relates to human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the administration scheme is adapted based on the subject's:
a) CSF pNFH concentration,
b) CSF, blood, serum or plasma NFL concentration, or
c) CSF, blood, serum or plasma NFM concentration.

The present invention also relates to the use of human interleukin-2 (IL-2) for the manufacture of a drug for use in the treatment of amyotrophic lateral sclerosis in a human subject, wherein each dose of human IL-2 administered to said subject during said treatment is between 0.1×10⁶ to 3×10⁶ international units (IU) and the administration scheme is adapted based on the subject's:
a) CSF pNFH concentration,
b) CSF, blood, serum or plasma NFL concentration, or
c) CSF, blood, serum or plasma NFM concentration.

The present invention also relates the use of human interleukin-2 (IL-2) in the treatment of amyotrophic lateral sclerosis in a human subject, wherein each dose of human IL-2 administered to said subject is between 0.1×10⁶ to 3×10⁶ international units (IU) and the administration scheme is adapted based on the subject's:
a) CSF pNFH concentration,
b) CSF, blood, serum or plasma NFL concentration, or
c) CSF, blood, serum or plasma NFM concentration.

The present invention also relates to a pharmaceutical composition comprising human interleukin-2 (IL-2) for use in the treatment of amyotrophic lateral sclerosis in a human subject, wherein each dose of human IL-2 administered to said subject is between 0.1×10⁶ to 3×10⁶ international units (IU) and the administration scheme is adapted based on the subject's:
a) CSF pNFH concentration,
b) CSF, blood, serum or plasma NFL concentration, or
c) CSF, blood, serum or plasma NFM concentration.

The present invention also relates to a method for treating amyotrophic lateral sclerosis in a human subject in need thereof, comprising:
a) Measuring CSF pNFH concentration; CSF, blood, serum or plasma NFL concentration; or CSF, blood, serum or plasma NFM concentration in a sample of the subject; and
b) Selecting a suitable administration scheme of human interleukin-2 (IL-2) for the subject based on the concentration measured in step a), wherein each dose of human IL-2 administered to said subject is between 0.1×10⁶ to 3×10⁶ international units (IU).

Indeed, instead of using low dose IL-2 therapy as disclosed above only in ALS subjects with a low to medium CSF pNFH concentration; CSF, blood, serum or plasma NFL concentration; or CSF, blood, serum or plasma NFM concentration, low dose IL-2 treatment may be used in the whole ALS population, but with distinct administration schemes depending on the subject's CSF pNFH concentration; CSF, blood, serum or plasma NFL concentration; or CSF, blood, serum or plasma NFM concentration.

In this context, for ALS subjects with a low to medium CSF pNFH concentration; CSF, blood, serum or plasma NFL concentration; or CSF, blood, serum or plasma NFM concentration (any embodiment disclosed in section above concerning the treatment of this subgroup of patients), an administration scheme close to that used in MIROCALS clinical trial may be used, with repeated cycles of human IL-2 administration, in particular cycles of 3 to 7 consecutive days of once-daily sub-cutaneous injection of 0.1×10⁶ to 3×10⁶ IU of human IL-2, preferably 0.2×10⁶ to 3×10⁶ IU of human IL-2, 0.3×10⁶ to 3×10⁶ IU of human IL-2, 0.4×10⁶ to 3×10⁶ IU of human IL-2, more preferably 0.5×10⁶ to 3×10⁶ IU of human IL-2, 0.6×10⁶ to 3×10⁶ IU of human IL-2, 0.7×10⁶ to 3×10⁶ IU of human IL-2, 0.8×10⁶ to 3×10⁶ IU of human IL-2, 0.9×10⁶ to 3×10⁶ IU of human IL-2, 1×10⁶ to 3×10⁶ IU human IL-2, 0.5×10⁶ to 2×10⁶ IU of human IL-2, 1×10⁶ to 2×10⁶ IU of human IL-2, in particular 0.5×10⁶ IU of human IL-2, 1×10⁶ IU of human IL-2, or 2×10⁶ IU of human IL-2 every 3 to 5 weeks (such as every 4 weeks) may be used.

For ALS subjects with a high CSF pNFH concentration; CSF, blood, serum or plasma NFL concentration; or CSF, blood, serum or plasma NFM concentration (any embodiment disclosed in section above concerning the treatment of this subgroup of patients), the cumulative dose of human IL-2 administered will preferably be higher. This may be obtained by different administration schemes:
a) In the case of repeated cycles of human IL-2 administration, an increased cumulative dose may be obtained by increasing the duration of each cycle (i.e. the number of consecutive days during which human IL-2 is administered for each cycle), increasing the frequency of the cycles (i.e. reducing the duration between two successive cycles, or by increasing both the duration and frequency of the cycles.
For instance, regarding duration of the cycles, instead of 3 to 7 days cycles, 7 to 10 days cycles may be used.
Regarding frequency, instead of cycles that are administered every 3 to 5 weeks, cycles that are administered every 1 to 3 weeks, more preferably every 1 or 2 weeks may be used.
b) Another type of administration scheme that may be particularly suited to ALS patients with a high CSF pNFH concentration; CSF, blood, serum or plasma NFL concentration; or CSF, blood, serum or plasma NFM concentration (any embodiment disclosed in section above concerning the treatment of this subgroup of patients) is the continuous (also referred to “metronomic”) administration.
This type of administration indeed permits to increase the cumulative dose while keeping each individual dose of human IL-2 administered lower than 3×10⁶ IU, preferably lower than 2×10⁶ IU, in order to preserve safety.
In particular, a pump permitting continuous administration of low dose human IL-2 (similar to those used for delivering insulin to diabetic subjects) may be used for ALS patients with a high CSF pNFH concentration; CSF, blood, serum or plasma NFL concentration; or CSF, blood, serum or plasma NFM concentration (any embodiment disclosed in section above concerning the treatment of this subgroup of patients).

It should also be noted that the CSF pNFH concentration; CSF, blood, serum or plasma NFL concentration; or CSF, blood, serum or plasma NFM concentration may be measured not only before starting low dose human IL-2 treatment, but also during follow-up, in order to monitor therapeutic efficiency. If the ALS patient has an initial low to medium CSF pNFH concentration; CSF, blood, serum or plasma NFL concentration; or CSF, blood, serum or plasma NFM concentration (any embodiment disclosed in section above concerning the treatment of this subgroup of patients) and a corresponding administration scheme as disclosed above has been administered to him/her but his/her CSF pNFH concentration; CSF, blood, serum or plasma NFL concentration; or CSF, blood, serum or plasma NFM concentration is later found to be high, then the administration scheme may be changed to one of the administration schemes disclosed above for subjects with high CSF pNFH concentration; CSF, blood, serum or plasma NFL concentration; or CSF, blood, serum or plasma NFM concentration.

### Uses and Methods for Stratification of Therapeutic Efficiency

The results of MIROCALS clinical trial also show that the CSF pNFH concentration; the CSF, blood, serum or plasma NFL concentration; or the CSF, blood, serum or plasma NFM concentration may be a useful biomarker for stratification of ALS subjects at randomization in a clinical trial intended to assess the therapeutic efficiency of a candidate ALS treatment, including human IL-2 and particularly low dose human IL-2 (i.e. each dose of human IL-2 administered to the ALS subject is kept low, between 0.1×10⁶ to 3×10⁶ international units (IU), see section above regarding human IL-2 doses to be used in the context of the present invention).

On this basis, the present invention also relates to the use of the CSF pNFH concentration; the CSF, blood, serum or plasma NFL concentration; or the CSF, blood, serum or plasma NFM concentration of a cohort of ALS patients included in a clinical trial intended to assess the therapeutic efficiency of a candidate ALS treatment (including human IL-2 and particularly low dose human IL-2, see above) as a biomarker for stratification of the cohort of ALS patients into three subgroups with low, medium or high concentration as defined herein, and separate randomization of each subgroup into the candidate ALS treatment or placebo arm.

The present invention also relates to a method for determining the therapeutic efficiency of a candidate ALS treatment (including human IL-2 and particularly low dose human IL-2, see above) in a cohort of ALS patients, comprising:
a) measuring the initial CSF pNFH concentration; the CSF, blood, serum or plasma NFL concentration; or the CSF, blood, serum or plasma NFM concentration of the cohort of ALS patients before administration of the candidate ALS treatment, and
b) stratifying the cohort of ALS patients with low, medium or high concentration as defined herein and separately randomizing each subgroup into the candidate ALS treatment or placebo arm.

Preferably, the candidate ALS treatment (including human IL-2 and particularly low dose human IL-2, see above) is tested in a double-blind, placebo-controlled trial, and the method comprises:
a) measuring the initial CSF pNFH concentration; the CSF, blood, serum or plasma NFL concentration; or the CSF, blood, serum or plasma NFM concentration of the cohort of ALS patients before administration of the candidate ALS treatment,
b) stratifying the cohort of ALS patients with low, medium or high concentration as defined herein and separately randomizing each subgroup into at least two arms, a first arm intended to receive the candidate ALS treatment and a second arm intended to receive a placebo,
c) administering placebo or the candidate ALS treatment to the cohort of ALS patients in a double-blind randomized manner until completion of the treatment or unacceptable toxicities,
d) after completion of the treatment, unblinding the cohort of ALS patients, and analyzing therapeutic efficiency of the candidate ALS treatment in all ALS patients, in one or more subgroups defined by their initial CSF pNFH concentration; the CSF, blood, serum or plasma NFL concentration; or the CSF, blood, serum or plasma NFM concentration, or in all ALS patients and in one or more subgroups defined by their initial CSF pNFH concentration; the CSF, blood, serum or plasma NFL concentration; or the CSF, blood, serum or plasma NFM concentration.

In the above uses and methods, the same assays and thresholds as in other uses and methods disclosed above may be used.

The project leading to this application has received funding from the European Union's Horizon 2020 research and innovation programme under grant agreement No 633413.

The following examples merely intend to illustrate the present invention.

### EXAMPLES

### Example 1: Mirocals: Modifying Immune Response and Outcomes in ALS (Mirocals)

Following the very positive data obtained in the IMODALS trial, in particular concerning the significant drop in plasma concentration of CCL2, a marker of disease progression, and the change in polarization of macrophages from an inflammatory M1 phenotype towards an anti-inflammatory and repairing phenotype M2, a new phase II trial referred to as “MIROCALS” has been launched to confirm the positive effect of low dose human IL-2 on ALS.

### Patients and Methods

The “Modifying Immune Response and OutComes in ALS” project (MIROCALS) tests in a proof-of concept/proof of mechanism (PoC/PoM) study the hypothesis that ld IL-2-treatment results in decreased rate of neuronal damage in ALS patients, as measured by early change in CSF neurofilament and CCL2 levels, and that this early change is predictive of long-term clinical efficacy as assessed by survival over an 18 months treatment period. A scheme presenting MIROCALS set-up is presented in FIG. 1.

### Design

This is a double-blind, randomized, stratified (on country and site of onset) placebo-controlled, parallel group, study of low dose IL-2 at 2 MIU/day for 5 days every 4 weeks during 18 months. The study is preceded by a three-month run-in period to establish safety and stability of the riluzole treatment.

### Treatments

All treatment packages consisting in 51 ml prefilled polypropylene syringes containing either 0.6 ml of a 2 MIU aldesleukine solution or 0.6 ml of 5% glucose water for injection are prepared, Labelled and packaged in a central pharmacy in aseptic and temperature controlled conditions. In case of poor tolerance PIs are allowed to prescribe flexible dose down 1MIU (0.3 ml) or 0.5MIU (0.15 ml) to control for patient compliance.

### Primary End-Points

The Primary efficacy outcome was time to death (survival) from any cause at 21 months (640 days) post-randomization in the ITT population

### Secondary End-Points

Long-term safety of ld IL-2 therapy over 18 months of treatment, efficacy on functional decline (ALSFRS, Vital Capacity), and core biomarkers including Tregs in blood through immuno-cytometry expressed as number and as percent of CD4 cells as markers of target engagement by treatment, CCL2 (plasma and CSF, through ELISA) as markers of inflammation, and pNFH levels (plasma and CSF, through ELISA) as a quantifier of neuronal damage.

### Ancillary Studies

New biomolecular targets for drug intervention through genomics and transcriptomics profiling of ld IL-2 responder and non-responder patients.

### Measurements

Core laboratory measures (pNFH and CCL2 in CSF and blood, and blood immunocytometry) are performed in a central lab under GCLP conditions. Additional supportive immuno-inflammatory markers and omics investigations are performed in Academic laboratories. For the measure of pNFH in CSF and blood, the principle of the assay is a sandwich enzyme immunoassay (EIA) for the quantitative measurement of human phosphorylated neurofilament H (pNF-H). The assay will be performed according the current version manufacturer's protocol of the BioVendor manual pNF-H kit (cat #RD191138300R; version 98 090911 20). In the first step the samples are added to duplicate wells on the plate pre-coated with chicken polyclonal anti-pNF-H antibody and incubated 60 minutes at room temperature (RT). Captured pNF-H is detected by incubation for 60 minutes at RT with rabbit polyclonal anti-pNF-H antibody labeled with horse-radish-peroxidase (HRP). Subsequently substrate is added. The catalytic conversion of the substrate by peroxidase is stopped after 15 minutes with stop solution. The optical density is measured photometrically at 450 nm/reference 630 nm (acceptable range: 550-650 nm). pNF-H concentration of human plasma and CSF samples is calculated from regression function of the standard curve run on the same plate. Note that the measured concentration of samples calculated from the standard curve must be multiplied by their respective dilution factor, because samples have been diluted prior the assay.

For the measure of NFL in CSF and blood, a SIMOA® assay was used, as in Kuhle J. et al., Clin Chem Lab Med. 2016; 54:1655-1661. The Simoa NfL assay was established using the NF-light assay ELISA kit from UmanDiagnostics (UmanDiagnostics, Umea, Sweden), transferred onto the Simoa platform with a homebrew kit (Quanterix Corp, Boston, MA, USA), and detailed instructions can be found in the Simoa Homebrew Assay Development Guide (Quanterix). In short, paramagnetic carboxylated beads (Quanterix) were activated using 1-ethyl-3-(3-dimethylaminopropyl) carbodiimide

(EDAC) (Quanterix) by adding 5% (v/v) 10 mg/mL EDAC to a magnetic beads solution with 1.4×106 beads/μL. Following a 30-min incubation at room temperature (RT), the beads were washed using a magnetic separator, and an initial volume, i.e. EDAC+ beads solution volumes in the previous step, of 0.3 mg/mL ice cold solution of the monoclonal capture antibody (mAB47:3, UmanDiagnostics) was added. After a 2-h incubation on a mixer (2000 rpm, Multi-Tube Vortexer, Allsheng, China) at RT, the beads were washed and an initial reaction volume of blocking solution was added. After three washes, the conjugated beads were suspended and stored at 4 °C pending analysis. The monoclonal detection antibody (1 mg/mL, mAB2:1, UmanDiagnostics) was biotinylated by adding 3% (v/v) 3.4 mM EZ-Link™ NHS-PEG4-Biotin (Quanterix) followed by a 30-min incubation at RT. Free biotin was removed using spin fi-tration (Amicono Ultra-2, 50 kDa, Sigma) and the biotinylated antibody was stored at 4 °C pending analysis. The assay was run on a Simoa HD-1 instrument (Quanterix) using a 2-step Assay Dilu-tion 2.0 protocol using 25 μL conjugated beads, 75 μL diluent [PBS; 0.1% Tween-20; 2% BSA; 10 mcg/mL TRU Block (Meridian Life Sci-ence, Inc., Memphis, TN, USA)], 20 μL biotinylated antibody, and 25 μL sample (or calibrator), which was followed by a 47 cadances incubation (1 cadance=45 s). After washing, 100 μL of streptavidin-conjugated β-galactosidase (Quanterix) was added, followed by a 7-cadence incubation and a wash. Prior to reading, 25 μL Resorufi β-D-galactopyranoside (Quanterix) was added. The calibrator curve was constructed using the standard from the NF-light assay ELISA (NF-Light®, UmanDiagnostics). Calibrators were run in duplicates and obvious outlier calibrator replicates were masked before curve fitting. Samples were diluted 4 fold and run in singlicates. Results have been compensated for the dilution. Two QC levels were run in duplicates in the beginning and the end of each run.

For QC with concentration 8.6 pg/mL, repeatability was 4.1% and intermediate precision was 5.6%.

For QC with concentration 96.6 pg/mL, repeatability was 3.8% and intermediate precision was 6.1%.
Validated Reference Range=1.9-1800 pg/mL
LLOQ=1.9 pg/mL

### Patients Inclusion Criteria

Run-in period selection: De novo patients, Possible, Probable, or Laboratory-Supported Probable, or Definite ALS by El Escorial Revised ALS diagnostic criteria, disease duration≤24 months, a vital capacity≤70% of normal, no prior or present riluzole treatment, signed informed consent.

RCT period: same criteria except, disease duration≤27 months, “no prior or present riluzole treatment” replaced by “stable on riluzole treatment for 3 months”.

### Patient Exclusion Criteria

Contra indication to lumbar puncture; other life-threatening disease; other disease precluding functional assessments; cancer within the past 5 years (except stable non-metastatic basal cell skin carcinoma or in situ carcinoma of the cervix); severe cardiac or pulmonary disease; documented auto-immune disorders except asymptomatic Hashimoto thyroiditis, women of child bearing age without contraception or pregnant or breast feeding; any clinically significant laboratory abnormality (exception of cholesterol, triglyceride and glucose).

### Statistical Analyses

The primary efficacy analysis compares the treatment groups on survival using (i) a stratified log rank test, and (ii) following adjustment on prognostic factor candidates (Cox model analysis), including age, vital capacity, εt ALSFRS scores and core biomarkers. Levels of pNFH and CCL2 from randomisation to M4 (plasma and CSF), and immunocytometry parameters are to be analysed using variance/covariance analyses (ANOVA/ANCOVA). Analysis of repeated measures of function (ALSFRS and SVC) is performed with joint rank analysis methods for informative censored data.

### Number of Patients

Overall 216 patients are to be randomised with 108 patients per group to attain a power>0.80, at pα_{(2-sided)}=0.05, to detect at 21 months an absolute difference in death rate of 17% (placebo expected survival: 0.65 vs 0.82 in ld IL-2 group)—representing a 54% decrease in risk of death (RR ld IL-2/PLA=0.46). Given an attrition rate of 10% from eligible at selection to randomisation, about 240 patients are to be screened.

### Results

### Demographic and Clinical Variables in MIROCALS Population

220 participants have been recruited, 137 in France, 83 in UK.

IL2 and Placebo Groups were well balanced with no significant differences between IL2 and Placebo arms for:
Site of onset (bulbar vs limb)
Sex
Age at recruitment
Disease duration (median 10 months)
Time from diagnosis (median 1.4 months)
El Escorial Category (Possible ~11% vs Definite ~24%)
ALSFRS-R at recruitment/randomization
SVC % at randomisation

### Safety

Table 1 below shows Serious Adverse Event (SAE)—DRUG-RELATED and confirms that the low dose IL-2 treatment is well tolerated.

TABLE 1
Serious Adverse Event (SAE) - DRUG-RELATED.

| | Placebo | IL2 | |
|---|---|---|---|
| | N = 110 | N = 110 | Total |
| SAE-MEDDRA SOC | N | N | N |
| Infections and infestations | 0 | 2 | 2 |
| Nervous system disorders | 0 | 1 | 1 |
| Skin and subcutaneous tissue disorders | 0 | 2 | 2 |
| Immune system disorders | 1 | 1 | 2 |
| Neoplasms benign, malignant and | 1 | 0 | 1 |
| unspecified (incl cysts and polyps) | | | |
| Respiratory, thoracic & | 1 [1†] | 0 | 1 |
| mediastinal disorders | | | |
| Gastrointestinal disorders | 1 | 0 | 1 |
| [†] outcome death | 4 | 6 | 10 |
| *The Clear-cell renal-cell carcinoma | 4 | 5 | 9 |
| was declared as SUSAR. Was also | 3.6% | 4.5% | 4.1% |
| declared as misdiagnosed case | | | |
| (paraneoplasic Σ) | | | |

The good tolerance of the treatment is confirmed by the analysis of patients who withdrew treatment during the study (Table 2 below). There were 47 patients overall in equal number in both treatment group. The main reason for stopping treatment was mainly related to disease progression as is commonly the case in ALS trials. This is also illustrated by (i) the relatively high compliance (66%) and (ii) the poor survival of patients stopping the treatment (38%) compared to the overall ITT population (59%). Only 4% of the overall population stopped treatment due to drug related adverse event.

TABLE 2
Reason of study drug discontinuation in the MIROCALS study

| | Placebo | | IL-2 | | All | |
|---|---|---|---|---|---|---|
| Reason Drug withdrawal | (n = 110) | | (n = 110) | | (n = 220) | |
| Total (percent overall) | 23 | (21%) | 24 | (22%) | 47 | (21%) |
| Disease progression | 15 | (14%) | 14 | (13%) | 29 | (13%) |
| Drug related | 3 | (3%) | 5 | (5%) | 8 | (4%) |
| Other | 5 | (5%) | 5 | (5%) | 10 | (5%) |
| number of cycles - median [range] | 11 | [1-19] | 10 | [1-18] | 10 | [1-19] |
| Treatment duration (days) -median [range] | 289 | [28-505] | 255 | [1-476] | 284 | [1-505] |
| Compliance (% trt duration/time | 68% | [5-97] | 64% | [0-94] | 66% | [0-97] |
| in study)- median [range] | | | | | | |

| | | | |
|---|---|---|---|
| Percent overall survival @ 640 days | 35% | 42% | 38% |

### Target Engagement in Blood—Tregs (Number & % CD4), CCL2

Statistical analysis of target engagement in blood, i.e. Tregs (Number & % CD4 in blood) and plasma CCL2 concentration of completers at week 18 (5^{th} cycle) is presented in Table 3 below:

TABLE 3
Target engagement in blood, i.e. Tregs (Number & % CD4 in blood)
and plasma CCL2 concentration of completers at week 18 (5^{th} cycle).
COMPLETERS @ W 18 - Maximum IL2 response in blood at 5th Cycle

| | | Randomisation | | Week 18 | | ANOVA REPEATED |
|---|---|---|---|---|---|---|
| Blood parameter | Treatment | (RDN) | | (W 18) | | MEASURES |
| (% overall patients) | groups | Mean (sd) | | Mean (sd) | | p value |
| Number of Tregs | Pla (N = 90) | 38.0 | (16) | 35.6 | (15) | Time × Treatment: |
| (84%) | IL2 (N = 94) | 37.4 | (14) | 157.7 | (70) | 8.5E−68 |
| % of Tregs among | Pla (N = 90) | 5.3 | (1.5) | 4.9 | (1.5) | Time × Treatment: |
| CD4 T cells | IL2 (N = 94) | 5.3 | (1.7) | 13.33 | (4.2) | 5.E−95 |
| (84%) | | | | | | |
| Plasma-CCL2 | Pla (N = 97) | 304 | (57) | 304 | (54) | Time × Treatment: |
| (87%) | IL2 (N = 95) | 309 | (65) | 228 | (50) | 1.5E−38 |

Pla: placebo arm. IL2: IL-2 arm.

These results show that MIROCALS clinical trial confirms the finding of previous clinical trial IMODALS that:
Tregs number and percentage are expanded by the human low dose IL-2 treatment, and
Plasma CCL2 concentration (illustrative of monocyte activation) is decreased by the human low dose IL-2 treatment.
   Disease Core Biomarker Response in CSF (pNFH, CCL2)

Table 4 below presents statistical analysis of the result of treatment on CSF biomarkers pNFH and CCL2 for completers at week 17.

TABLE 4
Statistical analysis of the result of treatment on CSF
biomarkers pNFH and CCL2 for completers at week 17.
COMPLETERS @ W 17 (trough level of 4th cycle in CSF)

| | | Randomisation | Week 17 | ANOVA REPEATED |
|---|---|---|---|---|
| CSF parameter | Treatment | (RDN) | (17) | MEASURES |
| (% overall patients) | groups | Mean (sd) | Mean (sd) | p value |
| CSF-pNFH | Pla (N = 87) | 2489 (1711) | 2365 (1577) | Time × Treatment: |
| (78%) | IL2 (N = 85) | 2357 (1453) | 2281 (1336) | NS |
| CSF-CCL2 | Pla (N = 87) | 593 (147) | 617 (143) | Time × Treatment: |
| (78%) | IL2 (N = 85) | 577 (163) | 586 (162) | NS |

Pla: placebo arm. IL2: IL-2 arm. NS: non significant.

These results show no treatment effect detected on core biomarkers in the CSF. This might be due to:
Insufficient sensitivity of markers,
Choice of Time of sampling (trough level vs max and/or too early in follow-up), or
A mixture of both.

### Treatment Effects on Survival (Unadjusted Univariate and Adjusted Multivariate Analysis)

Statistical analysis of treatment effects on survival (log-rank) is presented in Table 5 below.

TABLE 5
Unadjusted univariate (log-rank) statistical analysis
of treatment effects on survival (log-rank).

| UNADJUSTED | | Hazard Ratio | |
|---|---|---|---|
| (Univariate) | n | [95% CI] | p value |
| Treatment IL2 | 220 | 0.81 [0.54-1.22] | 0.33 |

The unadjusted log rank analysis of the primary survival endpoint showed a 19% decrease in the risk of death (HR[95% CI]=0.81 [0.54-1.22]) in the IL-2LD treated group compared to placebo (Table 5); As the study was not powered to detect this degree of change, this effect did not reach statistical significance.

The suggested treatment effect is however supported by the adjusted analysis showing a statistically significant reduction in risk of death in the ld IL2 treated group.

An unsupervised stepwise Cox multivariate analysis selected 5 covariates as significantly and independently predicting survival (Table 6 below):
A demographic variable: “Age” at inclusion;
A clinical variable: “ALSFRS-R at randomisation”; and
Three core biomarker parameters measured at randomisation: “CSF-pNFH levels”; “Number of Tregs”;” “Plasma CCL2 levels”.

In the adjusted analysis, the “treatment” factor and “treatment by CSF-pNFH interaction” were statistically significant. No other treatment by factor interaction was found significant (Table 6).

TABLE 6
ITT population—results of the Cox multivariate stepwise selection model

| | | | | | | | | |
|---|---|---|---|---|---|---|---|---|
| ALSFRS-R @ RND | 220 | −0.155 | 0.8563 | 0.0189 | −8.21 | 2.3 E−16 | 0.825 | 0.8886 |
| AGE | 220 | 0.029 | 1.029 | 0.0124 | 2.31 | 0.021 | 1.004 | 1.0543 |
| CSF-pNFH @ RDN | 220 | 0.00017 | 1.00017 | 6.4E−05 | 2.61 | 0.0091 | 1.00004 | 1.00029 |
| NTREG @ RDN | 220 | −0.0178 | 0.9824 | 0.0075 | −2.36 | 0.018 | 0.968 | 0.9970 |
| Plasma-CCL2 @ RDN | 220 | 0.0033 | 1.0033 | 0.0019 | 1.77 | 0.077 | 0.9996 | 1.0069 |
| Treatment-IL2 | 220 | −1.153 | 0.31579 | 0.427 | −2.70 | 0.00696 | 0.1367 | 0.7294 |
| Treat by CSF- | 220 | 0.00034 | 1.00034 | 0.0001 | 3.26 | 0.0011 | 1.00014 | 1.00054 |
| pNFH_interaction | | | | | | | | |

The treatment effect when adjusted on prognostic covariates shows a significant 68% decrease in risk of death in the IL-2_{LD} group (Hazard ratio [95% CI]: 0.32 [0.14-0.73]; p<0.007, when no pNFH is present), and a significant treatment by CSF-pNFH level interaction (HR=1.00034, p=0.0011), indicating that the magnitude of the treatment effect decreases with increasing CSF-pNFH level.

This suggests that a decreasing treatment effect is associated with increasing disease “aggressiveness” as indicated by the HR of pNFH.

To test for the robustness of this result, we performed several sensitivity analyses.

A Cox model including only the treatment, CSF-pNFH and the interaction of both showed a very similar result (see Table 7 below) as compared with the full model (Table 6 above), ruling out “overfitting” issues.

TABLE 7
Adjusted multivariate (Cox model) statistical analysis
of treatment effects on survival (log-rank).

| ADJUSTED | | Hazard Ratio | |
|---|---|---|---|
| (Multivariate) | n | [95% CI] | p value |
| TREATMENT-IL2 | 220 | 0.27 [0.12-0.62] | 0.0017 |
| CSF-pNFH @ RDN | 220 | 1.0002 [1.0001-1.0003] | 2.5E−05 |
| INTERACTION | 220 | 1.0004 [1.0001-1.0006] | 7.1E−05 |

CSF-pNFH adjusted analysis shows a statistically significant 70% decrease in the risk of death over the trial period for the IL2 group vs Placebo (Table 7).

We further checked the consistency of the results with an independent measure of CSF-pNFH level as measured at Inclusion, 12 to 18 weeks before the measure at randomisation used in the primary model (Table 8 below).

TABLE 8
ITT population—Cox model including Treatment factor, CSF-pNFH at inclusion and interaction.

| Parameters ITT analysis | n | coef | z | Pr(>\|z\|) | exp(coef) | lower .95 | upper .95 |
|---|---|---|---|---|---|---|---|
| TREATMENT-IL2 | 220 | −0.8021 | −2.12 | 0.035 | 0.448 | 0.213 | 0.944 |
| CSF-pNFH_IMP @ INC | 220 | 0.00022 | 4.57 | 4.8E−06 | 1.00022 | 1.00013 | 1.00032 |
| TREATMENT-IL2 X CSF- | 220 | 0.00023 | 2.55 | 0.011 | 1.00023 | 1.00005 | 1.00040 |
| pNFH_interaction | | | | | | | |

Again a similar trend for the treatment and treatment by pNFH interaction was found supporting the robustness of the findings.

Post-Hoc analysis to explore the treatment by CSF-pNFH interaction, identified 19 participants (9% of the overall ITT population) with CSF pNFH levels below the lower limit of quantification (LLOQ: 0-750 pg/ml). As all were alive at follow-up cut-off (640 days), no treatment effect could be determined in this group.

On the right side of the CSF-pNFH level distribution (see FIG. 2) we determined a threshold of 3700 pg/ml that we used to stratify the population into High (n=47, 21% of the overall ITT population) vs Low and Medium (n=154, 70% of the overall ITT population) CSF-pNFH levels at randomisation.

Of note the hypothesis of normal distribution of the CSF-pNFH level distribution was rejected (p=2.2×10⁻¹⁶ by the Shapiro-Wilk test), and the distribution found multimodal by the Ameijeiras-Alonso test (see FIG. 2). The threshold of 3700 pg/ml separating High CSF-pNFH from Medium and Low CSF-pNFH is located between the first and second density peaks of the multimodal distribution (see FIG. 2).

In order to interpret the interaction between treatment efficacy and CSF pNFH concentration, the survival probability in time of subjects treated with IL-2 with low CSF pNFH concentration (below the sandwich ELISA LLOQ of 750 pg/ml), medium CSF pNFH concentration (between the ELISA LLOQ and a cut-off value) and high CSF pNFH concentration (above the cut-off value) was analyzed with iterative testing of cut-off, and the best distinguishing cut-off was found to be about 3700 pg/ml in the specific sandwich ELISA used.

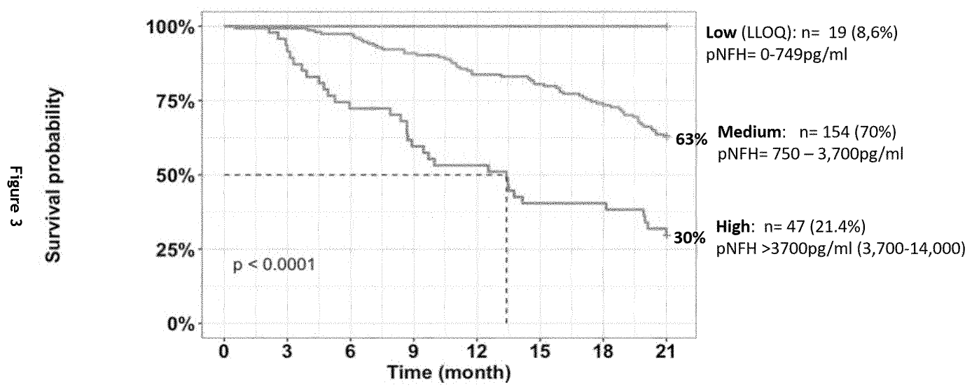

FIG. 3 presents the survival probability in time of the three groups of patients with the best cut-off of 3700 pg/ml for distinguishing medium and high CSF pNFH concentration, and clearly shows the therapeutic efficacy of low human IL-2 treatment in subjects with low or medium CSF pNFH concentration before IL-2 treatment.

As the mean CSF pNFH concentration in ALS subjects of the IL-2 arm at randomization was about 2350 pg/ml (see Table 4 above), the first threshold value (750 pg/ml) between low and medium CSF pNFH is about one third (0.319) of the mean CSF pNFH concentration, while the second threshold value (3700 pg/ml) between medium low and high CSF pNFH is about 1.6 times (1.574) the mean CSF NFL concentration.

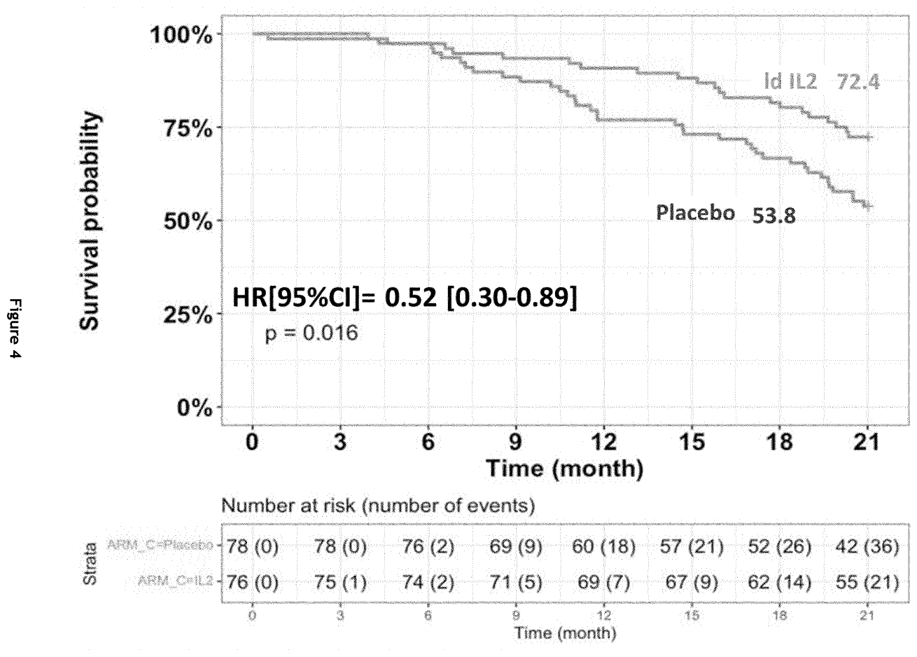

Log rank test of the treatment effect in the Medium and High pNFH level groups showed a marked difference in treatment efficacy with a significant treatment effect favouring the IL2L_{d} group in the Medium pNFH population (70% of ITT population; HR 95% CI]=0.52 [0.30-0.89], p=0.016) (FIG. 4).

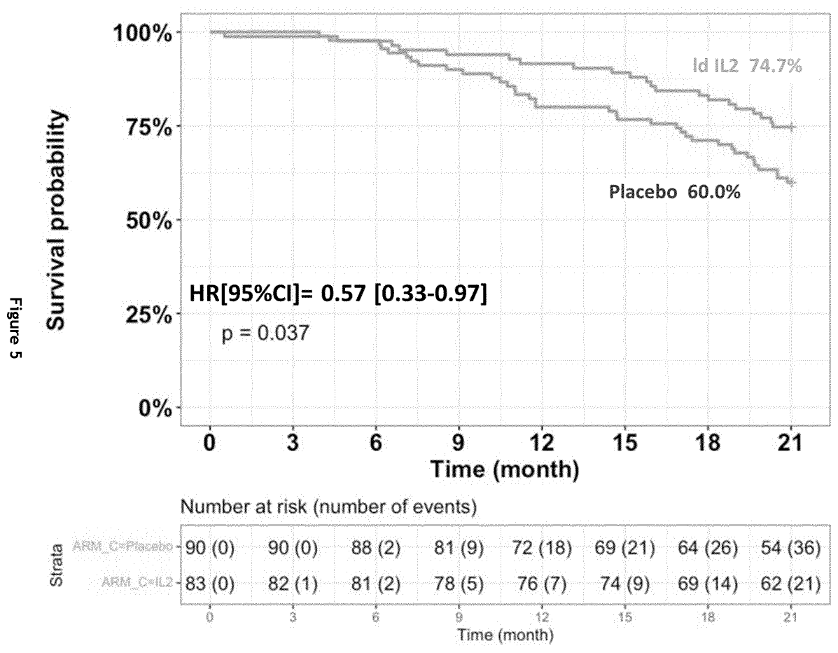

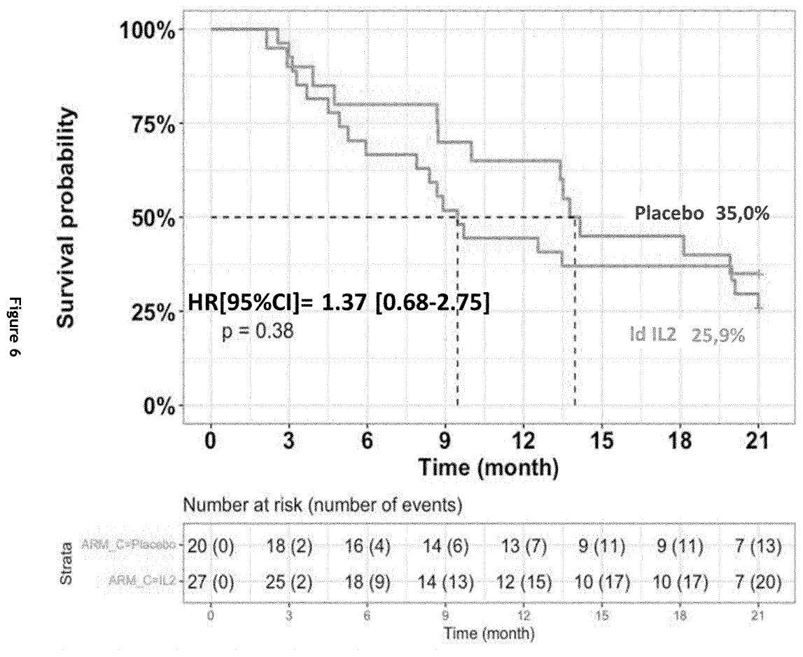

When pooling LLOQ and Low groups (achieving 79% of the ITT overall population) the treatment effect remained significant (HR [95% CI]=0.57 [0.33-0.97], p=0.037) (FIG. 5). However, an increase in the risk of death in the ld IL2 treated group is suggested in the High CSF-pNFH group, with an imbalance in early deaths, but a similar survival at the end of the follow-up period, with the log-rank test not reaching significance level (21% of ITT population, (HR [95% CI]=1.37 [0.68-2.75], p=0.38) (FIG. 6).

Table 9 below provides a summary of IL2 effect on survival according to CSF-pNFH levels at randomisation (univariate cox model).

TABLE 9
Summary of IL2 effect on survival according to CSF-
pNFH levels at randomisation (univariate cox model).

| | | Hazard Ratio | |
|---|---|---|---|
| Population | N (% trial) | [95% CI] | p. value |
| Medium pNFH | 154 (70%) | 0.52 [0.30-0.89] | 0.0175 |
| Low (LLOQ) + | 173 (79%) | 0.57 [0.33-0.97] | 0.039 |
| medium pNFH | | | |
| High pNFH | 47 (21%) | 1.37 [0.68-2.75] | 0.38 |
| All | 220 (100%) | 0.81 [0.54-1.22] | 0.33 |

These results show that the treatment by CSF-pNFH interaction is quantitative.

At the two extremes of the CSF-pNFH distribution, no significant treatment effect can be detected.
In the Low (LLOQ) CSF pNFH category because there are no deaths in either group, and
In high CSF pNFH because progression rate is too rapid.

Altogether, the post hoc analysis, in line with the primary adjusted analysis, suggests that beyond a CSF-pNFH threshold—which we based on the CSF-pNFH distribution in our population, and defined at 3700 pg/ml CSF-pNFH using the particular measurement method used in MIROCALS—the treatment schedule of ld IL2 we used in the trial is not effective with regards to the very fast progression/high disease aggressiveness in the Highest CSF-pNFH level group, while for about 70-80% of the population below that threshold, ld IL2 treatment induced a significant decrease in risk of death of about 43-48% over the follow-up period (640 days).

Finally, ALSFRS-R score slopes of change unadjusted analysis showed a 14% decrease in rate of change in the ld IL2 group compared to the placebo group (median [range] rate of change in points/month):
Placebo: −1.11 [−13.57; +0.21], vs ld-IL2, −0.95 [−8.46; +4.34], which did not reach statistical significance by the joint rank test (Win ratio [95% CI]:1.17 [0.86-1,59], p=0.32).

As for survival analysis, pre-specified adjusted analysis on CSF-pNFH level (through ancova on win score) showed a strong prediction of CSF-pNFH level measured at randomisation on the rate of change of ALSFRS-R (F_{(1,216)}=22.02, p=4.8 10⁻⁶), no significant treatment effect (F_{(1,216)}=0.22, p=0.6), but a significant treatment by CSF-pNFH interaction (F_{(1,216)}=3.9, p=0.049).

Post-hoc analysis of this interaction using the same CSF-pNFH threshold as for survival analysis, essentially replicates the findings in survival with a statistically significant 24% decrease in rate of change in the low CSF-pNFH stratum for the ld IL2 group compared to placebo (Table 10).

Table 10 below provides a summary of joint-rank analysis for ALSFRS-R slope of change according to CSF-pNFH levels.

TABLE 10
Summary of joint-rank analysis for ALSFRS-R slope of change according to
CSF-pNFH levels (All Patients vs LOW vs MEDIUM vs LOW + MEDIUM vs HIGH).

| | ALSFRS-R | | | | Joint-Rank analysis | |
|---|---|---|---|---|---|---|
| | Slope of change (point/mth) | | | | Win ratio test | |
| CSF-pNFH level | | Placebo | | IL-2 | Win ratio | p |
| population | N1 | Median [range] | N2 | Median [range] | (95% CI) | value |
| Full trial pop. | 110 | −1.11 | 110 | −0.95 | 1.17 | 0.32 |
| all patients | | [−13.57; +0.21] | | [−8.46; +4.34] | (0.86−1.59) | |
| | | | | (14% decrease) | | |
| Low (LLOQ) | 12 | −0.38 | 7 | −0.37 | 0.83 | 0.73 |
| all patients | | [−1.27; 0.0] | | [−0.82; −0.02] | (0.28−2.47) | |
| Medium | 78 | −1.06 | 76 | −0.81 | 1.59 | 0.015 |
| all patients | | [−3.23; 0.03] | | [−4.2; 4.34] | (1.09−2.30) | |
| | | | | (24% decrease) | | |
| Low + medium | 90 | −0.95 | 83 | −0.78 | 1.41 | 0.054 |
| all patients | | [−3.23; 0.03] | | [−4.2; 4.34] | (0.99−2.00) | |
| | | | | (18% decrease) | | |
| High | 20 | −1.99 | 27 | −2.11 | 0.79 | 0.51 |
| UWLE | | [−13.57; 0.21] | | [−8.46; 2.17] | (0.40−1.57) | |
| | | | | (6% increase) | | |

Table 10 shows that low dose IL-2 therapy also significantly decreases the rate of functional deterioration, as assessed by the ALSFRS-R slope of change in subjects with medium CSF pNFH before treatment.

Correlation of CSF pNFH Concentration with CSF NFL Concentration

FIG. 7 shows the CSF pNFH concentration with respect to the CSF NFL concentration, and shows that CSF pNFH concentration is correlated with CSF NFL concentration, by regression formula CSF-NFL=725+2.9(CSF-pNFH).

As a result, corresponding low, medium and high levels of CSF NFL may be defined as follows:
Low CSF NFL: CSF NFL concentration lower than 2900 pg/ml (=725+2.9×750),
Medium CSF NFL: CSF NFL concentration comprised between 2900 pg/ml and 11455 pg/ml (=725+2.9×3700), and
High CSF NFL: CSF NFL concentration higher than 11455 pg/ml.

The mean concentration of CSF NFL in ALS subjects of the IL-2 arm at randomization was about 8250 pg/ml. As a result, the first threshold value (2900 pg/ml) between low and medium CSF NFL is about one third (0.349) of the mean CSF NFL concentration, while the second threshold value (11455 pg/ml) between medium low and high CSF NFL is about 1.4 times (1.388) the mean CSF NFL concentration.

Table 11 below presents treatment effects adjusted on CSF-NFL (multivariate Cox model), including interaction and shows that CSF-NFL concentration at randomization is also highly correlated to treatment efficiency.

TABLE 11
Treatment effects adjusted on CSF-NFL (multivariate
Cox model), including interaction.

| ADJUSTED | | Hazard Ratio | |
|---|---|---|---|
| (Multivariate) | n | [95% CI] | P value |
| CSF-NFL @ RDN | 220 | 1.0001 [1.0001-1] | 1.65e−07 |
| Treatment IL2 | 220 | 0.47 [0.22-1.006] | 0.0518 |
| INTERACTION | 220 | 1.0001 [1.0000-1] | 0.0186 |

### CONCLUSION

The MIROCALS study, a randomised double blind clinical trial, succeeded in recruiting a population of ALS patients at the earliest practicable time in the course of the disease. Indeed this “incident” population has until now been poorly documented as it represents only about one tenth of the “prevalent” population usually recruited in ALS trials (prevalent ALS population 1/10,000 vs incident ALS population 1-2/100,000). As such, for France and the UK combined (about 1,200-2,400 new patient/year), the sample screened (n=304) or randomised (n=220) represents a significant fraction of exhaustivity, meaning the results should have a reasonable reproducibility.

Recruiting newly diagnosed patients means that, at time of recruitment, patients have not yet fully expressed their full disease phenotype. Indeed to increase the probability of recruiting patients at the earliest time, “El-Escorial Criteria possible patients” were also included. These represented up to 11.4% of the overall population recruited (12.8% in the High CSF-pNFH group vs 9.7% in the low CSF-pNFH group). As a consequence, the MIROCALS population proved to be more representative of ALS phenotypic heterogeneity than most ALS trials that usually recruit over-selected prevalent patients. The wide heterogeneity characteristics of the MIROCALS population strengthens the generalisability of evidence resulting from the study.

Finally, the results are based on robust data with no loss to follow-up, complete traceability, and complete information on core biomarkers at randomisation, which minimises the risk of bias in relation to missing data.

Overall, results presented above show that the low-dose IL2 treatment is well tolerated and reduced the risk of death and rate of functional deterioration.

In addition, even though the unadjusted analysis of the treatment effect did not reach statistical significance either for the survival primary outcome or ALSFRS-R secondary clinical outcome, for both, adjusted analysis on the quantifier of neuronal damage CSF-pNFH showed that this biomarker is not only a strong and useful predictor of disease progression and survival (as known in the art), but also a treatment modifier, i.e. a predictor of the treatment effect.

CSF-pNFH has consistently been reported as a strong predictor of survival, which is why it was chosen as the core biomarker of disease “activity”. However, to the best of our knowledge, the above results show for the first time that CSF-pNFH is further a “treatment modifier”, i.e. predicting magnitude or direction of the treatment effect.

Considering the significant disease heterogeneity and complexity of ALS, at the design stage, it was planned to systematically check for a “treatment response modifying” factor in order to advance understanding of the disease, and the above results show that CSF-pNFH at inclusion or randomization is such a factor.

In particular, the overall adjusted global analysis showed a significant 73% decrease in the risk of death, with a treatment by pNFH level interaction. Accordingly, in the MIROCALS study, CSF-pNFH predicted that for high CSF-pNFH values over a threshold defined in our population and with a specific ELISA sandwich assay at 3700 pg/ml, ld IL2 does not have clinical efficacy, while below the threshold (70% of overall population) it decreases the risk of death by nearly 2-fold and functional disability progression rate by a quarter.

Post-hoc analysis of the interaction indeed showed in 70 (medium CSF pNFH) to 80% (low+medium CSF pNFH) of the trial population that:
IL2 decreases by 48% the risk of death over a 640 days (21 months) follow-up,
IL2 decreases the rate of functional deterioration, as assessed by the ALSFRS-R slope of change, by 24% (ie, 3.12 points/year) in the same population.

This effect can be interpreted as reflecting interaction with the degree of aggressiveness (“intensity”) of the disease process.

A similar interaction is evidenced for CSF NFL, which concentration is highly correlated to CSF pNFH, which is thus also a treatment modifier.

The above analysis is a prospective analysis and results of the adjusted analysis could theoretically be a chance finding through “overfitting”. This can however be ruled out in the MIROCALS study for the following reasons:
The CSF-pNFH at randomisation parameter was pre-specified as covariate for treatment adjustment as well as treatment by CSF-pNFH interaction in the protocol and the pre-hoc Statistical Plan, drafted before unblinding.

Our study design and statistical plan was indeed developed precisely to allow detection of a heterogeneous response to treatment using the unequivocal endpoint of survival, for which we achieved a 100% ascertainment;
The level of significance of the CSF-pNFH effect, treatment effect and of their interaction is convincing of a true effect;
The same effects were retrieved in an analysis limiting the covariates to treatment, CSF-pNFH and its interaction with treatment (3 covariates for 90 events);
The same effects were retrieved with an independent CSF-pNFH measure done at inclusion; and
The same interaction was retrieved with an independently measured functional parameter, ie, ALSFRS-R rate of functional disability progression.

Moreover, the fact that the MIROCALS population well represents the entire ALS incident population makes us confident that the CSF-pNFH threshold we defined (3700 pg/ml in the MIROCALS ALS population using a specific ELISA sandwich assay) can be used for future drug trial stratification when recruiting newly diagnosed patients.

### BIBLIOGRAPHIC REFERENCES

Ahmadzadeh M, Rosenberg S A. Blood 2006; 107:2409-14
Alexianu M E et al. Neurology 2001; 57: 1282-9;
Appel S H et al. Arch Neurol 1988; 45: 381;
Beers D R et al. JCI Insight. 2017; 2(5):e89530;
Beghi E et al. Neurology 2000; 54: 469-469;
Beghi E, et al. The epidemiology and treatment of ALS: focus on the heterogeneity of the disease and critical appraisal of therapeutic trials. Amyotroph Lateral Scler. 2011 January; 12(1):1-10. doi: 10.3109/17482968.2010.502940
Bensimon G et al N Engl J Med 1994; 330: 585-91
Camu W. et al., Repeated 5-day cycles of low dose aldesleukin in amyotrophic lateral sclerosis (IMODALS): A phase 2a randomised, double-blind, placebo-controlled trial, eBioMedicine, Volume 59, 2020, 102844;
Castela E et al. JAMA Dermatol 2014; 150: 748-51
Cudkowicz M E et al. Ann Neurol 2006; 60: 22-31;
DiBernardo A B et al. Biochimica et Biophysica Acta 1762 (2006) 1139-1149;
Drachman D B et al. Ann Neurol 1994; 35: 142-50;
Dupont G. et al. Cytokine. 2014 September; 69(1):146-9
Engelhardt J I et al. Arch Neurol 1993; 50: 30-6;
EP109748,
EP118617
EP136489,
EP200280,
Evans M C et al. Mol Cell Neurosci 2013; 53: 34-41;
Gille B et al. J Neurol Neurosurg Psychiatry. 2019 December; 90(12):1338-1346;
Gordon P H et al. Lancet Neurol 2007; 6: 1045-53;
Hall E D, Oostveen J A, Gurney M E. Glia 1998; 23: 249-56
Hartemann A et al. Lancet Diabetes Endocrinol 2013; 1: 295-305
Henkel J S et al. EMBO Mol Med 2013; 5: 64-79
Hensley K et al. J Neurochem 2002; 82: 365-74
Ito S et al. Mol Ther J Am Soc Gene Ther 2014. 22: 1388-95
Kawamata T et al. Am J Pathol 1992; 140: 691; Engelhardt J I et al. Arch Neurol 1993; 50: 30-6;
KOEN POESEN ET AL: “Diagnostic and Prognostic Performance of Neurofilaments in ALS”, FRONTIERS IN NEUROLOGY, vol. 9, 18 Jan. 2019 (2019-01-18)
Koreth J et al. N Engl J Med 2011; 365: 2055-66
Malek T R, Bayer A L. Nat Rev Immunol 2004; 4: 665-74
Mantovani S et al. J Neuroimmunol 2009; 210: 73-9;
McGeer P L et al. Muscle Nerve 2002; 26: 459-70;
Miller R G, et al. Cochrane Database of Systematic Reviews 2012, Issue 3. Art. No.: CD001447
PRADO LAURA DE GODOY ROUSSEFF ET AL: “Longitudinal assessment of clinical and inflammatory markers in patients with amyotrophic lateral sclerosis”, JOURNAL OF NEUROLOGICAL SCIENCES, ELSEVIER SCIENTIFIC PUBLISHING CO, AMSTERDAM, NL, vol. 394, 4 Sep. 2018 (2018-09-04), pages 69-74
Rentzos M et al. Acta Neurol Scand 2012; 125: 260-4;
Saadoun D et al. N EngI J Med 2011; 365: 2067-77
SakaguchiS et al. Cell 2008; 133: 775-87
Sheean R K et al. JAMA Neurol. 2018; 75(6):681-689
Stommel E W et al. Amyotroph Lateral Scler 2009; 10: 393-404
Tan E et al. Arch Neurol 1994; 51: 194;
Tateishi T et al. J Neuroimmunol. 2010 May; 222(1-2):76-81
Thonhoff J R et al. Curr Opin Neurol 2018; 31: 635-9
Thonhoff J R et al. Neurol Neuroimmunol Neuroinflammation 2018; 5: e465
Troost D et al. Neuropathol Appl Neurobiol 1990; 16: 401-10;
U.S. Pat. No. 4,530,787;
U.S. Pat. No. 4,656,132;
U.S. Pat. No. 4,748,234
U.S. Pat. No. 4,752,585;
van den Berg L H et al. Neurology 2019; 92:e1610-e1623
WO2012123381A1,
WO2014023752A1,
WO2016025385A1,
WO2016164937A2
WO2021/176044
Zhao W et al. J Neuroimmune Pharmacol Off J Soc Neurolmmune Pharmacol 2013; 8: 888-99
Kuhle J. et al., Clin Chem Lab Med. 2016; 54:1655-1661
Gisslen et al., EBioMedicine. 2016; 3:135-140
Benatar et al., Neurology. 2020; 95:e59-69
Gaiottino et al., PLoS One. 2013; 8:e75091; Gille et al., Neuropathol Appl Neurobiol. 2019; 45:291-304
Norgren N et al. (2003) Brain Res 987: 25-31
Norgren N. et al. (2002) Hybrid Hybridomics 21: 53-59

1.-15. (canceled)

16. A method for treating amyotrophic lateral sclerosis in a human subject in need thereof, comprising:
a) measuring the concentration of p-NFH in a cerebrospinal fluid sample of the subject, or the concentration of NFL in a CSF, blood, serum, or plasma sample of the subject, or the concentration of NFM in a CSF, blood, serum, or plasma sample of the subject; and
b) administering to the subject:
i) human interleukin-2 (IL-2), wherein each dose of human IL-2 administered to said subject is 0.1×10⁶ to 3×10⁶ international units (IU) and optionally riluzole when:
the concentration of p-NFH in cerebrospinal fluid (CSF p-NFH) measured in step a) is low to medium,
the concentration of NFL in cerebrospinal fluid, blood, serum or plasma measured in step a) when measured by a specific assay is low to medium, or
the concentration of NFM in cerebrospinal fluid, blood, serum or plasma measured in step a) when measured by a specific assay is low to medium; or
ii) riluzole and optionally another treatment different from human IL-2 when:
the concentration of p-NFH in cerebrospinal fluid (CSF p-NFH) measured in step a) is high,
the concentration of NFL in cerebrospinal fluid, blood, serum or plasma measured in step a) when measured by a specific assay is high, or
the concentration of NFM in cerebrospinal fluid, blood, serum or plasma measured in step a) when measured by a specific assay is high.

17. The method of claim 16, wherein step b) comprises administering to the subject:
i) human interleukin-2 (IL-2), wherein each dose of human IL-2 administered to said subject is 0.1×10⁶ to 3×10⁶ international units (IU) and optionally riluzole when:
the concentration of p-NFH in cerebrospinal fluid (CSF p-NFH) measured in step a) is:
lower than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA),
lower than a CSF_pNFH_medium-high_other-assay threshold value that is linearly correlated to the CSF_p-NFH_medium-high_sandwich-ELISA threshold value, or
lower than a CSF, blood, serum, or plasma_NFL_medium-high_specific-assay threshold value that is linearly correlated to the CSF_p-NFH_medium-high_sandwich-ELISA threshold value,
the concentration of NFL in cerebrospinal fluid, blood, serum or plasma (CSF, blood, serum or plasma NFL) before human IL-2 administration is lower than a CSF, blood, serum, or plasma_NFL_medium-high_specific-assay threshold value that is linearly correlated to the CSF_p-NFH_medium-high_sandwich-ELISA threshold value, or
the concentration of NFM in cerebrospinal fluid, blood, serum, or plasma (CSF, blood, serum or plasma NFM) before human IL-2 administration is lower than a CSF, blood, serum, or plasma_NFL_medium-high_specific-assay threshold value that is linearly correlated to the CSF_p-NFH_medium-high_sandwich-ELISA threshold value; or
ii) nriluzole and optionally another treatment different from human IL-2 when:
the concentration of p-NFH in cerebrospinal fluid (CSF p-NFH) measured in step a) is:
higher than a CSF_p-NFH_medium-high_sandwich-ELISA threshold value of about 1.6 times the mean concentration of p-NFH in CSF samples in a cohort of ALS patients when measured by a sandwich enzyme-linked immunosorbent assay (ELISA),
higher than a CSF_p-NFH_medium-high_other-assay threshold value that is linearly correlated to the CSF_p-NFH_medium-high_sandwich-ELISA threshold value, or
higher than a CSF, blood, serum, or plasma_NFL_medium-high_specific-assay threshold value that is linearly correlated to the CSF_p-NFH_medium-high_sandwich-ELISA threshold value,
the concentration of NFL in cerebrospinal fluid, blood, serum or plasma (CSF, blood, serum or plasma NFL) before human IL-2 administration is higher than a CSF, blood, serum, or plasma_NFL_medium-high_specific-assay threshold value that is linearly correlated to the CSF_p-NFH_medium-high_sandwich-ELISA threshold value, or
the concentration of NFM in cerebrospinal fluid, blood, serum, or plasma (CSF, blood, serum or plasma NFM) before human IL-2 administration is lower higher a CSF, blood, serum or plasma_NFL_medium-high_specific-assay threshold value that is linearly correlated to the CSF_p-NFH_medium-high_sandwich-ELISA threshold value.

18. The method of claim 16, wherein step b) comprises administering to the subject:
i) human interleukin-2 (IL-2), wherein each dose of human IL-2 administered to said subject is 0.1×10⁶ to 3×10⁶ international units (IU) and optionally riluzole when the subject has:
a concentration of p-NFH in cerebrospinal fluid (CSF p-NFH) before human IL-2 administration lower than about 4500 pg/ml, or
a concentration of NFL in cerebrospinal fluid (CSF NFL) before human IL-2 administration lower than about 13800 pg/ml
ii) riluzole and optionally another treatment different from human IL-2 in other cases.

19. The method of claim 18, wherein:
the concentration of p-NFH in cerebrospinal fluid (CSF p-NFH) before human IL-2 administration is measured by the sandwich enzyme-linked immunosorbent assay (ELISA); or
the concentration of NFL in cerebrospinal fluid (CSF NFL) before human IL-2 administration is measured by a Single molecule array assay.

20. The method of claim 16, wherein step b) comprises administering to the subject:
i) human interleukin-2 (IL-2), wherein each dose of human IL-2 administered to said subject is 0.1×10⁶ to 3×10⁶ international units (IU) and optionally riluzole when the subject has:
a concentration of p-NFH in cerebrospinal fluid (CSF p-NFH) before human IL-2 administration about 500 to 4500 pg/ml, or
a concentration of NFL in cerebrospinal fluid (CSF NFL) before human IL-2 administration 2200 to 13800 pg/ml; or
ii) riluzole and optionally another treatment different from human IL-2 in other cases.

21. The method of claim 20, wherein:
the concentration of p-NFH in cerebrospinal fluid (CSF p-NFH) before human IL-2 administration is measured by the sandwich enzyme-linked immunosorbent assay (ELISA); or
the concentration of NFL in cerebrospinal fluid (CSF NFL) before human IL-2 administration is measured by a Single molecule array assay.

22. The method of claim 16, wherein when human IL-2 is selected as treatment in step b), it is administered as repeated injections of 0.1×10⁶ to 3×10⁶ IU of human IL-2.

23. The method of claim 22, wherein repeated cycles of 3 to 7 consecutive days of once-daily sub-cutaneous injection of 0.1×10⁶ to 3×10⁶ IU human IL-2 are administered to the subject.

24. The method of claim 23, wherein each cycle consists of 5 consecutive days of once-daily sub-cutaneous injection of 1×10⁶ to 3×10⁶ IU IL-2.

25. The method of claim 22, wherein the cycles are administered every 2 to 6 weeks.

26. The method of claim 16, wherein when human IL-2 is selected as treatment in step b), it is administered continuously according to a metronomic schedule.

27. The method of claim 16, wherein the treatment selected in step b) is administered for the life-time of the subject or until unacceptable drug-related serious adverse event.

28. The method of claim 16, wherein when human IL-2 is selected as treatment in step b), it is administered by subcutaneous or intravenous route.

29. The method of claim 16, wherein:
a) the treatment administered to the subject does not comprise the administration of regulatory T cells to the subject,
b) when human IL-2 is selected as treatment in step b), it is not complexed with anti-human IL-2 antibodies,
c) the treatment administered to the subject does not comprise the administration of rapamycin or any other suppressive agent of effector T cells (Teffs) to the subject, or
d) any combination of a) to c).

30. The method of claim 16, wherein when human IL-2 is selected as treatment in step b), step b) further comprises administering riluzole to said subject.

31. The method of claim 30, wherein riluzole is orally administered at a daily dose of 50 mg to 100 mg, taken in two equal doses of 25 mg to 50 mg separated by about 12 hours.

32. The method of claim 16, wherein when human IL-2 is selected as treatment in step b), it is a recombinant form of human IL-2.

33. The method of claim 32, wherein when human IL-2 is selected as treatment in step b), it is aldesleukin.

34. A method for treating amyotrophic lateral sclerosis in a human subject in need thereof, comprising:
a) measuring CSF pNFH concentration; CSF, blood, serum or plasma NFL concentration; or CSF, blood, serum, or plasma NFM concentration in a sample of the subject; and
b) selecting a suitable administration scheme of human interleukin-2 (IL-2) for the subject based on the concentration measured in step a), wherein each dose of human IL-2 administered to said subject is 0.1×10⁶ to 3×10⁶ international units (IU).

35. A method for determining the therapeutic efficiency of a candidate ALS treatment in a cohort of ALS patients, comprising:
a) measuring the initial CSF pNFH concentration; the CSF, blood, serum, or plasma NFL concentration; or the CSF, blood, serum, or plasma NFM concentration of the cohort of ALS patients before administration of the candidate ALS treatment, and
b) stratifying the cohort of ALS patients with low, medium, or high concentration as defined herein and separately randomizing each subgroup into the candidate ALS treatment or placebo arm.
